(19) United States
(12) Patent Application Publication
(10) Pub. No.: US 2026/0035652 A1
(43) Pub. Date: Feb. 5, 2026

(54) SYSTEMS AND METHODS FOR ENABLING OPTICAL BIOPROCESSES IN CELL MANUFACTURING

(71) Applicant: Cellino Biotech, Inc., Cambridge, MA (US)

(72) Inventors: Matthias Wagner, Cambridge, MA (US); Matthew Sullivan, Westwood, MA (US); Christopher Harrison, Cambridge, MA (US); Jean Carlos Serrano, Cambridge, MA (US); Jose Valdez, Cambridge, MA (US); Mohammad Imani Nejad, Cambridge, MA (US); Lukas Vasadi, Cambridge, MA (US); Sangkyun Lee, Newton, MA (US); Kien Tran, Cambridge, MA (US); Ed Tekeian, Cambridge, MA (US); Lydia Skrabonja, Cambridge, MA (US); Catherine Pilsmaker, Arlington, MA (US); Ozge Whiting, Cambridge, MA (US); Blair Morad, Cambridge, MA (US); Shuhang Wang, Cambridge, MA (US); Scott Luro, Somerville, MA (US); Joshua Blouwolff, Cambridge, MA (US); Alexander Stange, Somerville, MA (US); Maya Berlin-Udi, Acton, MA (US)

(21) Appl. No.: 19/285,320

(22) Filed: Jul. 30, 2025

Related U.S. Application Data
(60) Provisional application No. 63/677,287, filed on Jul. 30, 2024.
(60) Provisional application No. 63/677,271, filed on Jul. 30, 2024.
(60) Provisional application No. 63/677,268, filed on Jul. 30, 2024.
(60) Provisional application No. 63/689,115, filed on Aug. 30, 2024.

(51) Int. Cl. C12M 1/36; C12M 1/00; C12M 1/12; C12M 1/34; C12M 3/00

(52) U.S. Cl. CPC C12M 41/48; C12M 23/22; C12M 23/34; C12M 23/42; C12M 37/00; C12M 41/08; C12M 41/14; C12M 41/34; C12M 41/36

(57) ABSTRACT

Systems and methods are disclosed for enabling optical bioprocesses in cell manufacturing. A cell manufacturing platform comprises a cell culture cassette supporting a cell culture; an optical engine configured to: capture images of the cell culture; and optically remove cells from the cell culture; and wherein the cell culture cassette remains stationary and the optical engine moves relative to the cell culture cassette to capture the images and to remove cells.

### RELATED APPLICATIONS

This application claims the benefit of priority to U.S. Provisional Application No. 63/689,115, filed Aug. 30, 2024, U.S. Provisional Application No. 63/677,287, filed Jul. 30, 2024, U.S. Provisional Application No. 63/677,271, filed Jul. 30, 2024, and U.S. Provisional Application No. 63/677,268, filed Jul. 30, 2024, each of which is incorporated herein by reference in its entirety.

### BACKGROUND

Autologous induced pluripotent stem cell (iPSC)-derived regenerative cell therapies that utilize the patient's own cells offer a promising avenue for addressing a variety of medical conditions. They minimize immune-related complications, and are exceptionally well-suited to meet the needs of an aging and increasingly diverse patient population.

Despite significant medical advantages to patients, the current artisanal cell manufacturing methods of generating autologous iPSC-derived therapies face substantial challenges in terms of scalability. These issues pertain to cost-effectiveness and volume production, rendering autologous cell therapies economically unfeasible. Currently, iPSCs are manufactured in high-grade clean rooms by scientists over 10-12 weeks. Somatic cells, like skin fibroblasts or blood cells, are isolated and then cultured in dishes. Scientists introduce reprogramming factors, such as Oct4, Sox2, Klf4, and c-Myc, to these cells, often using Sendai viral vectors. Once pluripotent colonies form, researchers carefully pick and transfer these colonies under a microscope to new culture dishes for an extensive stabilization and vector clearing phase, followed by expansion and harvest. Resulting iPSCs are subjected to quality control (QC) release testing and cryopreserved for long-term storage.

This manual process is time-consuming and expensive, and in its current form cell therapies would only be available to those who are able to pay the significant cost involved. In addition, many cell therapy providers would have a difficult time even getting regulatory approval because of scaling issues in the late clinical trial stages. In addition, manual processes may be subject to many delays due to quality control issues, staffing issues, and other problems. Thus, there is a need in the art for scalable, automated or semi-automated solutions to enable wide-scale and efficient adoption of regenerative cell therapies, and these solutions must address the myriad technical challenges involved in automating a multi-month process performed by skilled technicians.

### BRIEF SUMMARY

According to certain aspects of the present disclosure, systems and methods are disclosed for enabling optical bioprocesses in cell manufacturing.

In an exemplary embodiment, a cell manufacturing platform, comprises a cell culture cassette configured to support a cell culture; an optical engine configured to: capture images of the cell culture; and optically remove cells from the cell culture; wherein the optical engine is configured to move relative to the cell culture cassette to capture the images and to remove cells without movement of the cell culture cassette.

In another exemplary embodiment, the optical engine comprises a first linear stage configured to move along a plane of the cell culture cassette; and at least one light source mounted on the first linear stage.

In a further exemplary embodiment, the first linear stage is further configured to move perpendicular to the plane of the cell culture cassette.

In yet another exemplary embodiment, the optical engine comprises a first linear stage configured to move in a first linear direction; a second linear stage mounted on the first linear stage, the second linear stage configured to move in a second linear direction perpendicular to the first linear direction; and at least one light source mounted on the second linear stage.

In another exemplary embodiment, the optical engine is further configured to move perpendicular to a plane of the cell culture cassette.

In a further exemplary embodiment, the optical engine further comprises one or more actuators to independently move the first linear stage and the second linear stage, thereby moving the at least one light source relative to the cell culture cassette.

In yet another exemplary embodiment, the cell culture cassette comprises a semi-transparent surface of a cell culture chamber.

In another exemplary embodiment, the semi-transparent surface comprises an optical film configured to enable imaging and cell removal by the optical engine.

In a further exemplary embodiment, the optical engine further comprises one or more sensors configured to measure optical power of the optical engine; and a platform manager is configured to adjust the optical power of the optical engine based on the measurements to adjust a precision of cell removal operations.

In yet another exemplary embodiment, the cell culture cassette is located on a first side of a transparent box and the optical engine is located on a second side of the transparent box.

In another exemplary embodiment, the platform is configured to adjust a laser energy based on a sterility requirement associated with the first side or the second side of the transparent box. In a further exemplary embodiment, the platform comprises a robotic system configured to move the cell culture cassette to and from the optical engine.

In yet another exemplary embodiment, the platform further comprises a plurality of cell culture cassettes, wherein the robotic system is further configured to move each of the plurality of cell culture cassettes to the optical engine.

In yet another exemplary embodiment, the optical engine comprises a laser light source configured to emit laser light to remove cells from the cell culture.

In another exemplary embodiment, the laser light source is configured to emit laser light at a wavelength between 700 nm and 1000 nm.

In a further exemplary embodiment, the optical engine further comprises an imaging system configured to capture brightfield and fluorescence images of the cell culture.

In yet another exemplary embodiment, the platform further comprises a platform manager configured to receive image data of the cell culture from the optical engine; analyze the image data to identify target cells for removal; and control the optical engine to remove the identified target cells.

In yet another exemplary embodiment, the platform manager is further configured to: generate a cell removal pattern based on the analyzed image data; and control the optical engine to remove cells according to the generated cell removal pattern.

In another exemplary embodiment, the cell culture cassette comprises a plurality of cell culture chambers, each chamber configured to support a separate cell culture.

In a further exemplary embodiment, the platform further comprises an environmental control system configured to maintain temperature, humidity, and gas composition within the cell culture cassette.

In an embodiment, a method of cell manufacturing comprises providing a cell culture cassette; adhering a cell culture within the cell culture cassette; capturing images of the cell culture by an optical engine; optically removing cells from the cell culture by the optical engine; and moving the optical engine relative to the cell culture cassette to capture the images and to remove cells without movement of the cell culture cassette.

### BRIEF DESCRIPTION OF THE DRAWINGS

FIG. 1 is a block diagram illustrating an autonomous cell manufacturing platform in accordance with various implementations.

FIG. 2 is a diagram illustrating an isolated cell imaging and scanning unit for use in a cell culture system in accordance with various implementations.

FIG. 3 is a diagram illustrating a laser system for use in a cell culture system in accordance with various implementations.

FIG. 4 illustrates various optical scan patterns for laser cell removal in accordance with various implementations.

FIG. 5 is a diagram illustrating an optical engine for use in a cell culture system in accordance with various implementations.

FIG. 6 is a diagram illustrating an optical system for use in a cell culture system in accordance with various implementations.

FIGS. 7A-C are plots representing a fiducial mark registration application in accordance with various implementations.

FIG. 8 is a diagram illustrating a laser system for use in a cell culture system in accordance with various implementations.

FIG. 9 is a diagram illustrating a cell imaging and editing system for use in a cell culture system in accordance with various implementations.

FIG. 10 is a diagram illustrating an example of sawtooth imaging acquisition in accordance with various implementations.

FIG. 11 is a flow chart of a method for performing continuous autofocus in a rapid imaging system in accordance with various implementations.

FIGS. 12A-D are diagrams illustrating a method of fabricating an encapsulated laser film in accordance with various implementations.

FIGS. 13A-C are diagrams illustrating a method for manufacturing substrate-embedded laser film in accordance with various implementations.

FIG. 14 illustrates a method for pre-conditioning of a laser film for cell culture process applications in accordance with various implementations.

FIGS. 15A-D illustrate a method for manufacturing a plasmonic film suitable for long-term cell culture in accordance with various implementations.

FIG. 16 is a diagram illustrating a method of constructing a cell culture surface in accordance with various implementations.

FIGS. 17A-C are diagrams illustrating methods for in situ laser monitoring in a cell culture system in accordance with various implementations.

FIGS. 18A-B are plots showing laser pulse and scattering detector signals in accordance with various implementations.

FIG. 19 is a diagram illustrating application of a laser pulse on a cell culture surface in accordance with various implementations.

FIG. 20 are images showing laser scanning of cell colonies in accordance with various implementations.

FIG. 21 is a diagram illustrating an imaging and image processing system for use in a cell culture system in accordance with various implementations.

FIG. 22 is a diagram illustrating a method of pre-patterning cell colonies in accordance with various implementations.

FIGS. 23A-V are diagrams illustrating an example series of cell culture process operations in accordance with various implementations.

FIGS. 24A-D are diagrams illustrating an example method of an in-place expansion sequence for a cell colony in accordance with various implementations.

FIGS. 25A-E are diagrams illustrating another example method of an in-place expansion sequence for a cell colony in accordance with various implementations.

These and other features of the present implementations will be understood better by reading the following detailed description, taken together with the figures herein described. The accompanying drawings are not intended to be drawn to scale. For purposes of clarity, not every component may be labeled in every drawing.

### DETAILED DESCRIPTION

The systems and methods disclosed herein include an autonomous cell manufacturing platform for efficient and scalable production of cells (e.g., iPSCs, differentiated cells) for use in cell therapies. The cell manufacturing platform utilizes optical bioprocesses such as optical imaging and optical-based cell culture management to continuously monitor and control cell culture processes without the need for constant human intervention. Cells are cultured, monitored, and managed in compact closed systems, such as closed cassettes, to provide a mobile, sterile cell culture environment while allowing for high multi-patient throughput in the overall system. The manufacturing platform also includes incubation spaces for cells to expand or grow over long periods of time, a fluid management system to inject and remove fluid media from the closed cassettes, and robotic elements to move the closed cassettes around the platform. The manufacturing platform utilizes artificial intelligence (AI) to analyze cell culture images and make determinations about cell interventions (e.g., cell removal, cell harvesting, media changes).

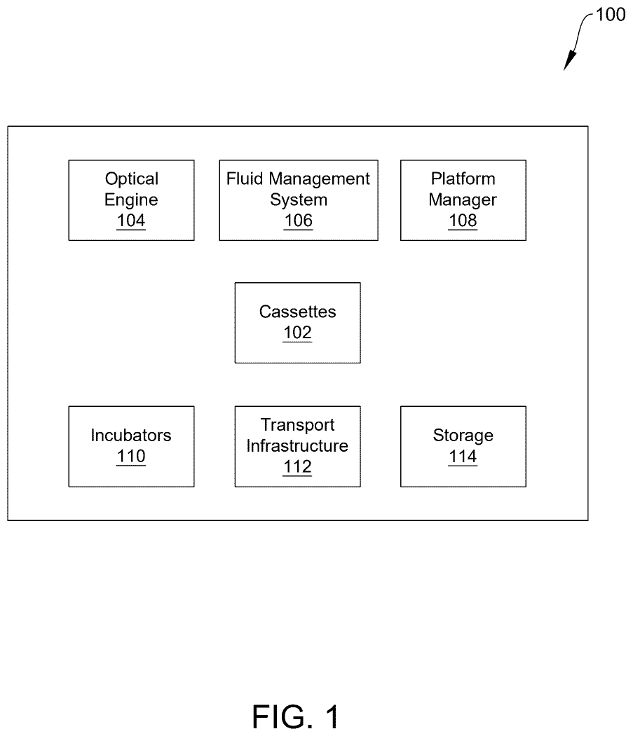

FIG. 1 is a block diagram illustrating an autonomous cell manufacturing platform 100 in accordance with various implementations. The platform 100 enables scalable, autonomous, and efficient production of cells for cell therapies. The platform 100 may provide a controlled, sterile environment for the manufacturing of cells for cell therapies. For example, the platform 100 may be a sealed enclosure with minimal interfaces to external environments and may be sterilized between uses. The platform 100 may be configured to adhere to various regulatory requirements for the production of cell and drug products administrable to patients.

Platform 100 includes a plurality of cassettes 102. Each cassette 102 may support a cell culture. The cell culture may start as source cells that undergo a cell culture process to produce an output cell product. For example, the source cells may be somatic cells that undergo reprogramming and expansion into output iPSC cells. In another example, the source cells may be iPSC cells that undergo differentiation and expansion into differentiated cells for use in cell therapies. Each cassette 102 may be a closed, sterile system that prevents contamination of the cell samples and allows for multi-sample and/or multi-patient processing using shared infrastructure.

Each cassette 102 may include one or more cell culture chambers, which may be closed fluidic chambers for growing cells (e.g., adherent cells). The cell culture chambers may include at least one transparent surface that includes an optical film upon which the cells adhere. The optical film may be flat and permanently attached to the first surface of the cell culture chamber. The optical film may be a multi-layered composition that includes various layers to enable selective light absorption, promote cell adherence, and prevent leaching of materials into the cell culture chamber. The optical film may enable optical-based label-free imaging and optical-based cell manipulation and removal techniques (e.g., using a laser). For example, the optical film may be configured to transmit wavelengths within certain wavelength ranges for imaging applications and at least partially absorb wavelengths in other wavelength ranges for optical removal applications. The cassettes 102 may be in a format that allows for observation of the cell culture at regular intervals. The cassettes 102 may also include various other components to enable autonomous optical processes, transport, and fluid exchange. For example, the cassettes 102 may include fiducials used to align imaging equipment, ports and tubing to enable fluid exchanges, and handles or other mechanical features to enable gripping, rotation, transport, or other physical manipulations of the cassette 102. In some implementations, the cassette 102 may have different designs and configurations for different purposes (e.g., expansion cassette, growth/maintenance cassette, differentiation cassette, harvesting cassette).

The platform 100 also includes an optical engine 104, which is configured to provide optical imaging and cell intervention functionality on the platform 100. Optical-based processes allow cell cultures to be monitored and managed without mechanical means, and thus not breaking the closed, sterile environment of the cassettes 102. The optical engine 104 may be located in a particular location within the platform 100, and cassettes 102 may be moved to the optical engine 104 from a storage location by robotic means for performing imaging and cell management functions. The optical engine 104 may be configured to provide label-free imaging suitable for long-term cell culture observation, although some implementations may include fluorescent imaging capability for immunofluorescent or other labeled images. The optical engine 104 may be configured to collect time-series images of cell cultures in the cassettes, which may be used by machine learning models to analyze cell growth, make predictions on future cell growth, and make determinations about interventions to perform on the cell culture.

The optical engine 104 may also be configured to function as a cell removal tool, or perform other methods of optical-based manipulation of the cell culture (e.g., cell poration, removal of ECM) in the cassettes 102. The optical engine 104 may be configured to target and remove cells at a regional, cluster-specific, and/or cell-specific level. Removal, in this context, may include selective destruction and/or removal of cells or cell regions, and non-destructive operations on cells (including intracellular delivery of compounds into cells or extraction of compounds from cells). The optical engine 104 may also be used to perform cell operations on a cell culture, such as splitting cell colonies into multiple sub-colonies, translating cell colonies across a cell culture surface, reducing confluence, surface area, or density of cell colonies by selective removal, and clonalization of cell colonies by repeated culling of portions of the cell colonies. In some implementations, the platform 100 may include more than one optical engine 104 that is shared by the cassettes 102.

One example implementation of the optical engine 104 is a laser-based system. The optical engine 104 may emit light within a first wavelength range for imaging cells in the cassettes 102. The optical engine 104 may also emit laser pulses within a second wavelength range that are designed to remove cells from the cell growth surface. Removal may be effectuated by, for example, heat transfer of energy from the laser pulses to the cells to dislodge/kill them, or conversion of optical energy into mechanical energy via the formation of microbubbles that kill and/or dislodge the cells.

The platform 100 also includes a fluid management system 106 that is configured to handle the injection and removal of fluids from the cassettes 102. Fluid media includes nutrients necessary for cells to grow, and cells expel waste into the fluid media. Thus, the fluid media must be periodically refreshed for cells to grow and be maintained in a healthy state. The fluid management system 106 may be located in a particular location within the platform 100, and cassettes 102 may be moved to the fluid management system 106 from a storage location by robotic means for performing fluidic exchange functions. The fluid management system 106 may include a receptacle for holding cassettes 102 in place during the fluid exchange. In some implementations, the receptacle may be configured to rotate, translate, or shake/vibrate the cassettes to achieve various fluid manipulation functions. The fluid management system 106 may also include tubing, pipetting, ports, and connectors to connect the cassettes with fluid and waste reservoirs. In some implementations, the fluid management system 106 is configured to aseptically connect to the cassettes to prevent contamination of the cell culture during fluid exchanges. In some implementations, the platform 100 may include more than one fluid management system 106 that is shared by the cassettes 102.

The platform 100 also includes a platform manager 108 configured to monitor and control the other components of the platform 100. The platform manager 108 may be, for example, a combination of on-premises and cloud computing resources that provide data collection, data analysis, and control functions. The platform manager 108 may be configured to gather data from a range of sources, organize the data in a manner that allows it to make predictions of success/quality/functionality of the cell culture, and in many cases do so on a cell-by-cell, cluster-by-cluster, or region-by-region basis. The platform manager 108 may utilize various artificial intelligence and machine learning models to monitor, analyze, and manage cell culture processes. The platform manager 108 may control the optical engine 104 according to cell management algorithms (for example, to maintain a certain cell density, to maintain certain exclusion areas within the cell culture container), in a timed manner (for example, delivering gene-activating or gene-editing compounds to cells at a specific interval), and/or as a result of predictions made by the platform manager 108 (for example, removal of cells predicted not to yield the desired phenotype or optimal level of function).

The platform 100 may also include one or more incubators 110. The incubators 110 may serve as storage locations for the cassettes 102 during cell growth, expansion, or maintenance, when the cassettes 102 are not being transported to the optical engine 104 or the fluid management system 106. The incubators 110 may be maintained at certain temperatures conducive for cell growth. The platform 100 may also include transport infrastructure 112 for moving cassettes 102 within the platform (e.g., from the incubators 110 to the optical engine 104 and back). The transport infrastructure 112 may include, for example, robotic arms that can grasp and move the cassettes, and/or rails that can transport the cassettes 102 from one location to another. The platform 100 may also include storage space 114, which may be used to store consumables within the platform. Such consumables may include, for example, fluids, pipettes, connectors, and other one-time use components. Such storage spaces may be temperature regulated, for example at 4° C. for the purpose of storing reagents or media. The platform 100 may include other components not illustrated in FIG. 1, such as interior/exterior interfaces for manual intervention (e.g., sterile glove ports) or for moving objects in and out of the platform 100 (e.g., load locks). The platform 100 may be built in an isolator model, in which the interior is aseptically separated from the external environment, or in the form of a biosafety cabinet, in which the interior is accessible but protected via airflows designed to prevent external contamination from entering. In some implementations, the platform 100 may include more than one optical engine 104 and/or fluid management system 106.

### Isolated Imaging and Scanning

In cell culture systems such as the ones described herein, there is a need to provide live-cell imaging and/or cell manipulation capabilities. However, any interaction with the cells involves a risk of cell contamination or cell damage. Cell samples should be kept in a sterile or patient-specific environment, potentially with environmental controls (e.g., temperature, humidity, gas concentration). The environment should be kept aseptic, with the ability to potentially sterilize it. Thus, there is a need to provide a sterile environment for cell cultures that still allows for imaging and cell manipulation.

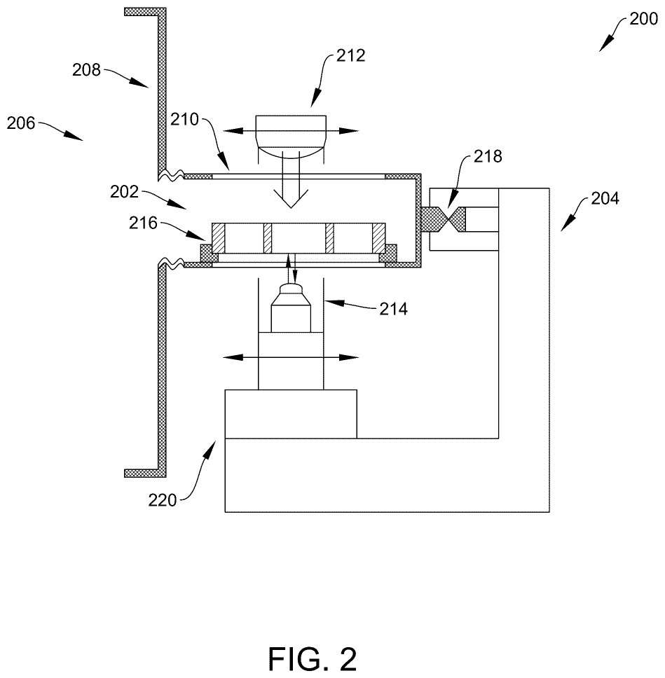

The systems and methods disclosed herein include an isolated cell imaging and scanning unit 200 as shown in FIG. 2. The unit 200 includes a combination of an optical compartment 202 and an optical engine 204. The interior of the optical compartment 202 is part of an aseptic cell processing environment 206, while the optical engine 204 is outside of the aseptic cell processing environment 206. A wall 208 attached to the optical compartment 202 separates the aseptic cell processing environment 206 from the outside environment. The wall 208 may include a small section of flexible material at the interface with the optical compartment 202. There are several advantages to the design shown in FIG. 2: (1) the optical equipment does not experience the conditions required for cell culture, which may include elevated temperature and humidity; (2) the optical equipment does not need to be sterilized, and therefore subjected to various chemicals; (3) the optical equipment may be exchanged or serviced without breaking open the biologically isolated environment; (4) the optical equipment may be shared between multiple isolators; and (5) the use of a moving optical assembly around a stationary, isolated biological sample minimizes impact on the cells from changes in environment or mechanical motions (e.g., sloshing, shocks).

Clear windows 210 on top and bottom of the optical compartment 202 allow transillumination from a light source 212 and imaging by an objective 214. Additionally, laser scanning to remove/treat cells is possible using the same objective 214 or another lens (or another optical assembly). The windows 210 may have integrated heaters to prevent or remove any condensation. A consumable 216 containing the cells may be locked into place in the optical compartment 202 via a variety of mechanisms. The optical compartment 202 itself may be temporarily mechanically locked to the optical engine 204 via an attachment mechanism 218 on both the optical compartment 202 and the optical engine 204. This lock mechanism ensure stability of the biological consumable 216 relative to the imaging and/or laser scanning paths. A motion control system 220 may be used to translate the imaging and/or scanning optics relative to the consumable 216. Fiducial markings on the consumable 216 may be used to establish X, Y, Z, and rotation of the consumable 216 versus the optical engine 204. In some implementations, the optical compartment 202 may also serve as the incubation compartment for the aseptic cell processing environment 206, allowing cells to be imaged and/or laser scanned without any movement of the consumable 216. In some implementations, the optical engine 204 may further include sensors (including but not limited to spectroscopic sensors) for measuring cells or cell media states. The unit 200 may contain multiple consumables that are transported via automated systems, or manually moved via gloves attached to the unit 200.

### Laser Astigmatism for Fast Autofocus

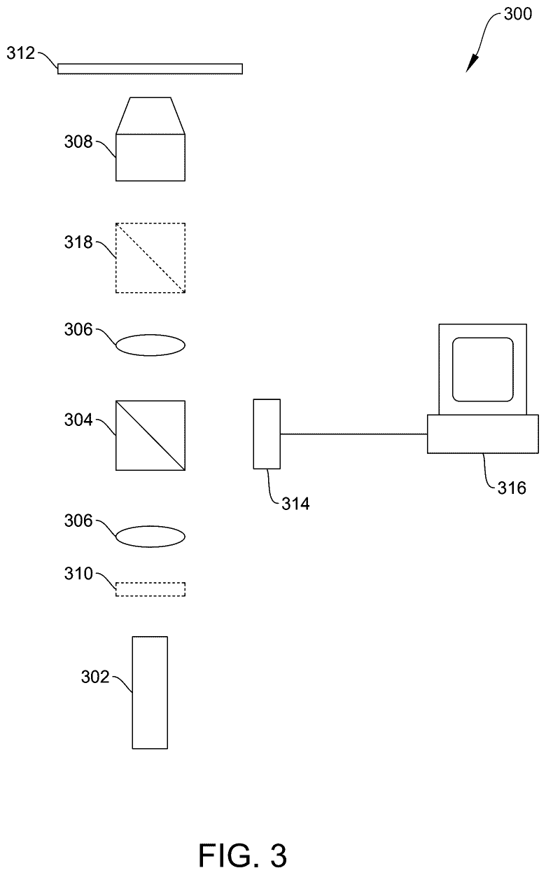

In a laser-based autofocus optical system, axial travel of a sample relative to the optical engine may result in inaccurate imaging results. Thus, there is a need in the art to limit the axial travel of a sample in an optical system. The systems and methods disclosed herein include a laser system 300 as illustrated in FIG. 3 that is designed to limit axial travel. The laser system 300 includes a laser source 302 combined with a beam splitter 304 and one or more lenses or lens groups 306 to expand and collimate a laser beam to fill the back aperture of an objective 308. Many inexpensive laser systems have a natural beam astigmatism that is appropriate, but additional cylindrical lenses or lens groups 310 may be used to obtain the desired laser astigmatism.

A sample 312 is moved relative to the objective 308, either by moving the sample 312 or the objective 308. Laser light reflects off the sample 312 and some of the light will be reflected onto a detector 314 that is connected to a processor 316. The detector 314 may be, for example, a camera sensor or a quadrant photodiode. In some implementations, it may be desirable to include a beam splitter 318 that allows separation of the autofocus and imaging system. When correctly aligned, the signal on the detector 314 will have minimum size and maximum brightness at focus, and moving the sample 312 through focus and looking for maximum brightness is a common technique for detecting autofocus. For beams with slight astigmatism, the brightness peak will be wider than optimal or even have two peaks. Computing the second moments of the brightness profile Mₓₓ, M_{xy}, and M_{yy} at each position (equivalent to computing the moment of inertia of an object, but in this case of a beam shape) allows determination of the major and minor axes of the beam at each position, either from an eigenvector decomposition or appropriate alignment of the laser source 302 and the detector 314. An astigmatism parameter may be calculated from the appropriately normalized difference between these two principal beam dimensions.

The system may be optically aligned such that the zero-crossing of this parameter coincides with the desired focus position and, with appropriate control of the focusing optics 304, 306, and 310, may be adjusted to allow for desired offsets. The use of a zero-crossing metric means that as few as two points, one on either side of focus, are necessary to confirm focus position, allowing for significantly shorter travel for an autofocus measurement. If the imaging and autofocus systems are separated, it is also possible to perform autofocus during the acquisition of multiple image planes. Using a zero-crossing metric has the additional advantage of allowing for closed-loop control based on feedback from the measurement at a single location. Such closed-loop focus control done in a band parallel to imaging or laser scanning may be used to continuously traverse a sample with an optical system to perform imaging and/or laser scanning operations while maintaining good focus, enabling high-throughput operation for cell culture imaging and/or laser-based cell processing.

### Optical Scan Strategies for Laser Cell Removal

When using a laser scanner as a cell removal tool, there is a danger that the energy imparted by the laser may cause unwanted effects on non-targeted cells and the growth surface (e.g., the optical film upon which the cells grow). Thus, there is a need in the art to determine an appropriate amount of laser energy and other parameters necessary to effectively remove target cells without overheating the substrate locally.

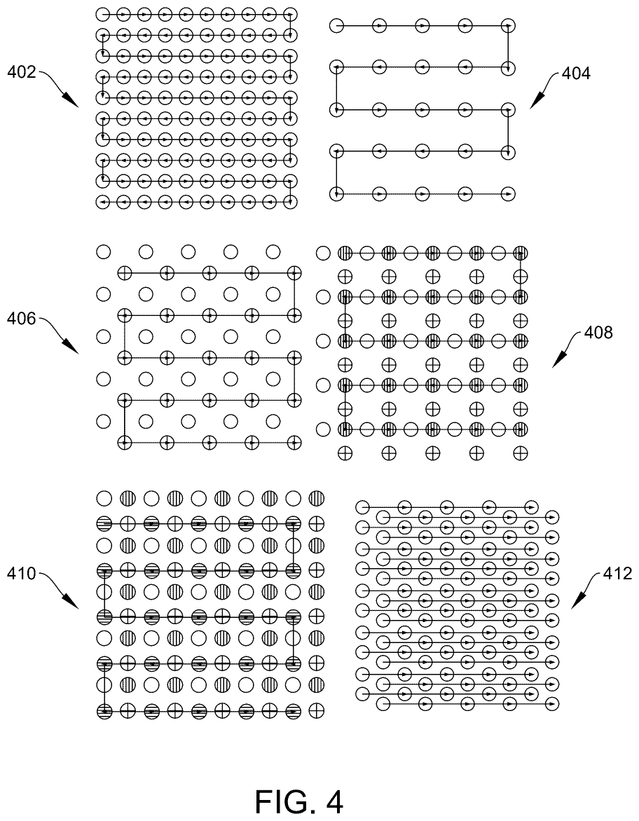

The systems and methods disclosed herein provide improved optical scan strategies for laser cell removal. The effectiveness of cell removal in a laser-based system increases as the density of laser hits increases. In such systems, the laser pulse rate is typically fixed and additional area is covered by raster scanning the position of the beam. FIG. 4 illustrates various optical scan patterns for laser cell removal in accordance with various implementations. For a given set of laser pulse parameters, a certain density of pulses in a given area as shown in pattern 402 may produce acceptable cell removal results. However, there are deleterious effects of laser scanning on the substrate and on cell adhesion that also scale with the density of scan points. These negative effects, particularly for pulses close to each other spatially, diminish more quickly with time than the cell removal effects. By temporally spreading out the pulses in a small region while still applying the same density of scan points, cell removal may be achieved while reducing the negative effects of heat.

An example implementation of an improved optical scan pattern is a staggered scan. An integer multiple of the original pulse spacing is chosen for an initial scan pattern 404. Subsequent scans 406, 408, and 410 are repeated using the same pattern, staggered slightly from the original by the original pulse spacing. In this implementation, nearest-neighbor points are separated in time by the amount of time needed to raster scan the entire pattern. Another implementation is to have a large spacing along the fast scan direction and correspondingly lower spacing along the slow scan direction, as shown in pattern 412. This may be particularly desirable in systems in which the location along the fast axis is reset after each line. Here, the nearest neighbor points are separated by the amount of time necessary to scan a single line.

Scanning patterns may have wider spacing along the fast scan axis, for example greater than 5 microns, greater than 10 microns, greater than 15 microns, or greater than 20 microns. Along the slow axis, the spacing may be decreased so as to hit close to points from a previous fast scan. For example, in the slow scan direction, the spacing may be less than 10 microns, less than 5 microns, less than 2.5 microns, or less than 1 micron. In some cases, the spacing along the fast scan axis (e.g., the spacing between subsequent laser pulses) is made greater than a multiple of the laser spot diameter as measured by 1/e² diameter, for example greater than 0.5× this diameter, greater than 1× this diameter, greater than 2× this diameter, or greater than 4× this diameter, so as to prevent thermal interactions. In some cases in which the laser interaction with an absorber causes the formation, growth, and collapse of microbubbles, the spacing of consecutive laser pulses may be done at some multiple of maximum bubble diameter, for example greater than 0.5× this bubble diameter, greater than 1× this bubble diameter, or greater than 2× this bubble diameter, to prevent bubble-bubble interactions.

### Laser Energy Pulse Stability Control on Moving Optical Platform

To properly manage cells with a cell removal tool (e.g., laser removal system) while avoiding damage to the cell substrate, precise control of the laser energy is necessary. Having a moving optical system relative to the cell substrate further complicates the cell removal process. Thus, there is a need in the art for methods to deliver uniform energy to a laser removal system with moving optics.

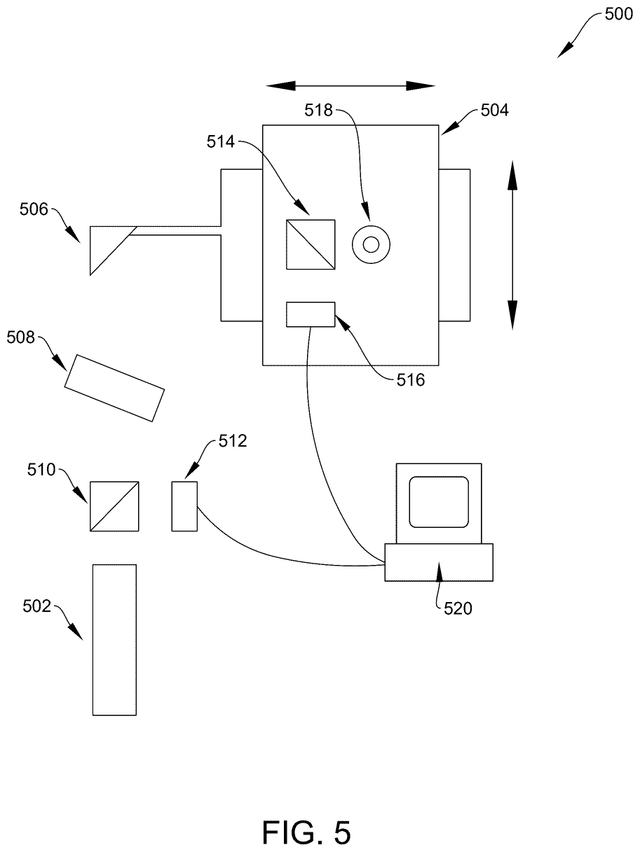

The systems and methods disclosed herein provide a method for stabilizing laser energy pulses on a moving optical platform. FIG. 5 is a diagram illustrating an optical engine 500 for use in a cell culture system in accordance with various implementations. The optical engine 500 includes a laser system 502 emitting light that is directed into a moving optical stage 504 using either a mirror 506 or some other method, such as an optical fiber. Before entering the optical stage 504, the laser is modulated using a modulator 508 (such as an acousto-optic modulator). Before modulation, a beam splitter 510 directs a small fraction of the laser energy to a power meter 512. After modulation, a second beam splitter 514 directs a small fraction of the modulated beam to a second power meter 516, as close as possible in the optical stage 504 to the laser removal target 518.

A calibration is done periodically to determine the fraction of energy striking the two power meters 512, 516 relative to the energy in the laser beam. During normal operation, information from the power meters 512, 516 is sent to a processor 520 and used to adjust the modulator 508 such that the output from the modulator 508 is equal to the desired output. The power meter 512 located before the modulator 508 is configured to compensate for fluctuations in the laser power. The processor 520 may account for the levels of modulation applied and may also account for changes in transmission that occur due to free space or fiber coupling losses that may change as the position of the optical stage 504 changes.

In some implementations, a stationary sensor in the plane of the sample may be used to measure the pulse energies or power through the entire system including the final objective or lens. In such implementations, the platform moves to the sensor position and a series of laser pulses is measured. In some cases, the sensor may be fitted with a pinhole aperture in the focal plane, allowing the shape of the laser beam spot in the sample plane to be profiled. The energy measured at this optical endpoint, during system startup or during calibration before laser-treating a sample, may be used in conjunction with the other built-in sensors to achieve and maintain accurate absolute pulse energy.

### Systems and Methods for Fiducial Marking, Alignment, and Calibration

Automated biological processes for adherent cells often employ imaging to monitor and control cell cultures. These images may be time-series images, and consumables are loaded and unloaded into imaging equipment repeatedly. In some cases, other spatially-specific optical operations, such as laser cell processing, spectroscopy, total internal reflection imaging, surface plasmon resonance imaging, or other modalities are employed. In some cases, images of the same consumable and cell culture are acquired on different optical instruments. This may be performed, for example, using a bank of identical optical instruments, or using optical instruments with different capabilities in conjunction with one another to provide complementary data or to acquire ground truth data for training deep learning models. Many of these operations require spatial alignment between images from different instruments, alignment of images over time, and alignment of images over different modalities. Additionally, many of these operations require repeatable positioning along the optical axis (e.g., z position) to produce consistent data over samples and time. Often, human judgment and intervention is required to correctly “focus” samples, even in automated microscopy systems, which can lead to additional variability. Moreover, variations in illumination and optical configurations cause errors in positioning of the imaging axes. Thus, there is a need in the art for effective calibration and alignment of optical systems.

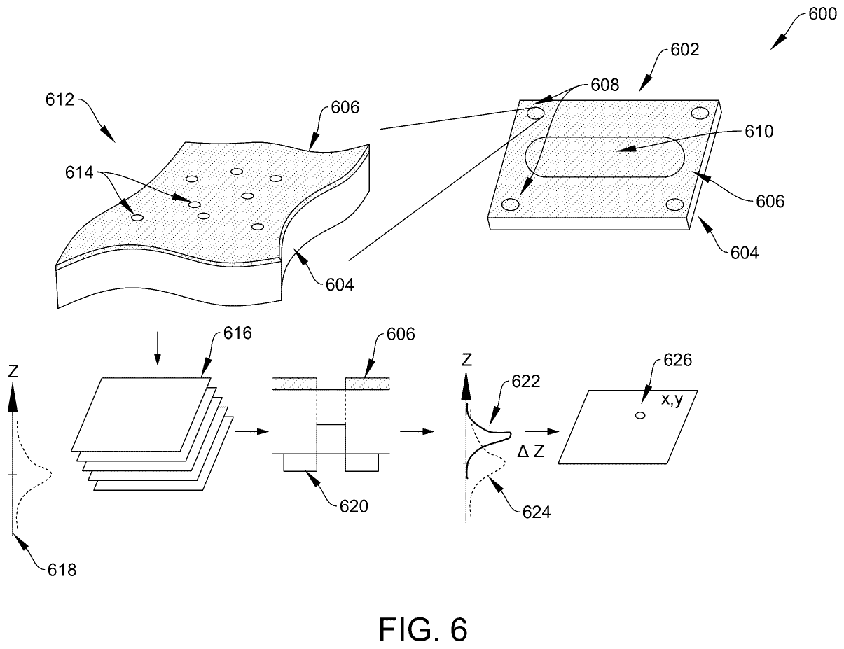

The systems and methods disclosed herein include the use of robust registration marks to acquire repeatable X, Y, and Z positioning in cell culture consumables, across timepoints and instruments. FIG. 6 is a diagram illustrating an optical system 600 for use in a cell culture system in accordance with various implementations. The optical system 600 includes a cell culture consumable 602. The cell culture consumable 602 may include, for example, a clear substrate 604 upon which a film 606 is deposited. The film 606 may be configured to change the transmission or reflection of incident light, and may cover most, or very little, of the consumable 602 surface. A series of registration marks 608 are marked into the film 606. The registration marks may be positive (e.g., a bump in the film 606), a negative (e.g., an absence of film 606), or a modification of the film's optical properties (e.g., transmission or reflection) for at least one wavelength. The registration marks 608 may be outside of a cell culture area 610 (as shown in FIG. 6), or may be within the cell culture area 610 in other implementations. The present implementations enable consistent acquisition of registration mark position even under conditions of high variability, such as when the registration marks 610 are in an active cell culture area, with cell growth, debris, extracellular matrix, etc. The registration marks 610 may be discrete marks, as shown in FIG. 6, or may extend over the entire consumable so as to provide local references at all points, and may further have patterns that indicate the absolute position along the surface.

FIG. 6 also shows an expanded view of a single registration mark 612, which in this implementation includes discrete puncta 614 patterned on the film 606 that is disposed on the clear substrate 604. The puncta 614 may be points of missing film, points of film surrounded by non-film areas, or points where the film properties have been changed (for example, made more or less transmissive or reflective, or more scattering, by some process). The process for creating the registration pattern may include, but is not limited to, laser marking, a lift-off process in which a temporary layer is patterned prior to film deposition and then used to selectively remove film in areas, a marking system that applies a dye or other material, or a material that is applied in solution or other means that nucleates into distinct points that remain intact upon the surface.

A feature of the present implementations is that the puncta 614 may be arranged in a random pattern, preferably in a “blue noise” pattern in which distances between points are consistent but the arrangement of points is random with respect to one another. This ensures that while image processing-based pattern-matching when aligned with the registration mark achieves a good match when aligned, there is a rapid drop-off in the registration signal when off-location, and there are no other semi-good matches. This is in contrast, for example, to crosshair or circular registration marks where the “goodness of match” to a template may fall off more slowly along certain axes, or there may be matches at off locations. Additionally, the use of puncta allows the widest range of marking systems to be used with consistent marks resulting. For example, pulsed laser systems may produce unpredictable results when drawing “lines” by superimposing multiple individual hits, but may consistently pattern standalone single hits into a film.

The overall size of the registration marks 608 may be made large enough such that local perturbations that may render individual puncta invisible are overcome by spanning a wider area. For example, if there are defects in the film 606, the registration marks 608 may be made several times larger than the maximum defect size. In other cases in which the mark is inside the cell culture area 610, the registration marks 608 may be made large enough to overcome local variations in cell culture images. On the other hand, it should be sufficiently small such that it fits reliably inside the field of view of the imaging instrument used to locate it, including a buffer to inherent misalignment with mechanical seating points.

The use of the registration marks 608 in a registration operation is also illustrated in FIG. 6. In the example shown in FIG. 6, a z-stack of transmission or reflection images 616 may be acquired in the registration mark area. Simultaneously, a non-imaging focus sensor may be used to acquire a surface focus signal 618. This non-imaging focus sensor may include, but not be limited to, a laser astigmatism-based sensor and method described herein, an LED-based system such as the Nikon® Perfect Focus system, or other systems and techniques that provide a reflection-based measurement of Z position that is not generated from the primary imager itself (i.e., “image-based focus”).

After image stack acquisition, a computing system attached to the imaging system applies a convolution with a 2D filter 620 (represented by a 1D cross-section in FIG. 6) with inside and outside portions that provide high contrast where puncta are present. The inside and outside portions (positive and negative coefficients, respectively, in this example) are sized to match the features in the film 606. The filter 620 may be adjusted to be robust under a degree of variation in the size of the puncta. After convolution, the absolute value of the resulting image may be used to be insensitive to the polarity of the marks. In some implementations, the resulting values may be divided by the absolute local intensity to produce signals that are insensitive to local spatial variations in illumination, or differences in illumination from instrument to instrument. Subsequent to this process, a correlation with a reference image of the registration mark is performed on each 2D z-plane. This correlation produces a match that is strong when the template aligns with the registration mark. Moreover, the signal may be highest when the registration mark is in focus.

The 2D filtering and image correlation may be combined into a single operation. The magnitude of the best template match is recorded for each Z plane as indicated by plot 622. The offset from the non-imaging focus method is also computed and shown as plot 624. This offset is then applied to subsequent imaging and other operations to assure consistent imaging and optical Z positioning across time and instruments, even when non-imaging focus systems perform differently. At the Z plane with the highest match value, the point 626 with the highest correlation match indicates the position of the registration mark. Thus, X, Y, and Z positioning and calibration may be performed robustly in a single pass.

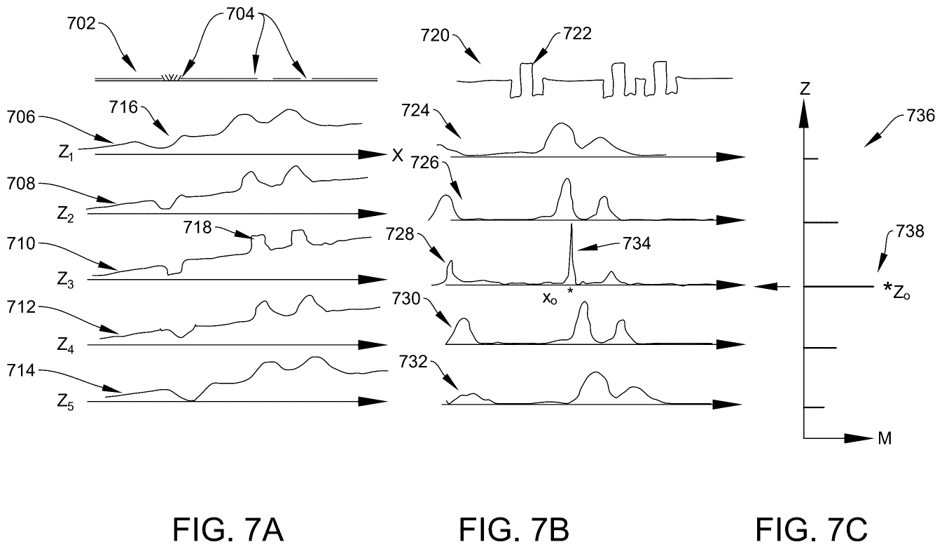

FIGS. 7A-C are plots representing a fiducial mark registration application in accordance with various implementations. The example shown is an X, Y, Z registration process, using a 1D representation of the system for explanatory purposes. FIG. 7A shows a film 702 with a fiducial marking pattern that includes puncta 704 in a pseudo-random arrangement patterned into the film 702. In this example, most of the points that have been patterned have increased the light transmission of the film at the imaging wavelength, but one has in fact reduced the light transmission of the film slightly. This may occur, for example, when a laser scanning system is used, and it completely ablates the film 702 in some areas, but simply damages it in others in a manner that increases light scattering. The film 702 may be, for example, a semi-transparent film designed to absorb laser pulses to process cell cultures. A series of images 706, 708, 710, 712, and 714 are taken at a range of Z heights in the area of the fiducial markings. In the first example Z plane image 706, there are some non-uniformities in background, which may be due to illumination, debris on the surface, film non-uniformities, and/or imaging system non-uniformities. In addition, it shows defocused features 716 corresponding to the puncta patterned into the film. As the film plane comes into focus, the features become sharper in area 718.

FIG. 7B shows an example image processing routine that a computing system connected to the imaging system may perform. A template 720 has a series of features 722 arranged in a pattern matching the fiducial marks. Each one of the features 722 is configured to compute a local “difference” corresponding to the difference between a hole the size of the puncta in the film and the surrounding film (or vice versa in a positive case). In the case that all puncta will have the same local polarity (bright on dark, or dark on bright), this local filter may be made part of the template 720 (e.g., by convolving it with the template image, which has 1s where the puncta should appear and 0s elsewhere), as shown in FIG. 7B. However, in instances in which the individual puncta may have different polarities, this filter should be run over the image first, then an absolute value applied to the resulting image, then the binary template correlated with this filtered/absolute valued image.

The purpose of the filter is to contrast the puncta against the local background, making the method insensitive to variations. The processed images, with filtering, absolute value, and template correlation, are shown for the Z planes in plots 724, 726, 728, 730, and 732. As shown by the individual traces, there is a local peak along the X axis where the template best matches the fiducial mark. This match is enhanced as the fiducial mark comes into focus, causing the highest peak 734 to appear after filtering and absolute value application. FIG. 7C shows a plot 736 illustrating the highest match value for each Z plane. The highest value is indicated by line 738, which corresponds to the best focus on the film plane. This position may be found by fitting the overall plot 736 or simply taking the maximum. After finding the Z max, the X (X, Y) max is located on that plane as indicated by the peak 734.

### Coupled Stage-Laser Motion for Continuous Laser Cell Removal

Optical methods may be used to image, monitor, and manipulate cell culture systems using optical-based cell culture containers (e.g., a closed cassette with transparent surfaces). In these systems, there is a need in the art to determine how to increase the throughput of a laser cell removal system for a given pulse repetition rate (PRR).

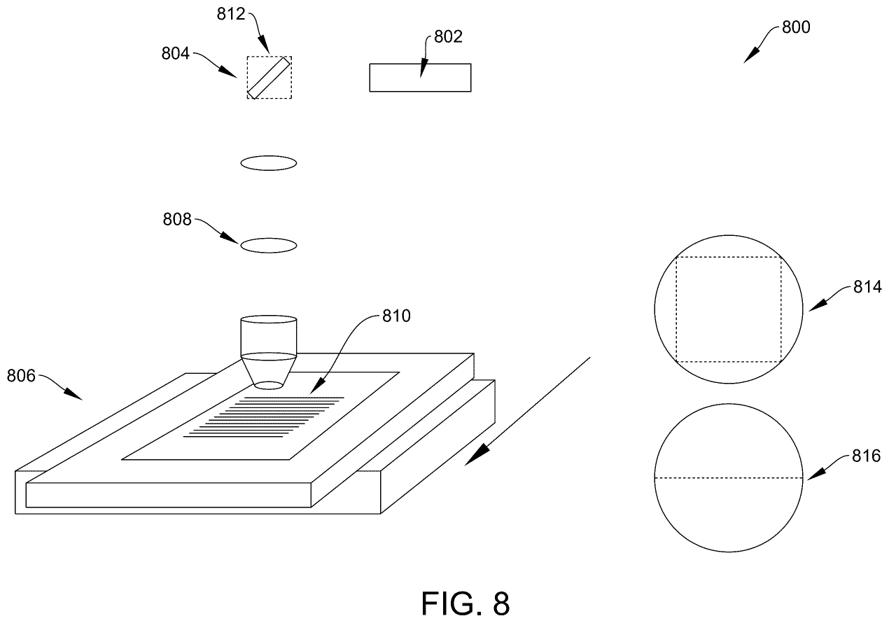

Systems and methods disclosed herein include methods for achieving a coupled stage-laser motion for continuous laser cell removal. FIG. 8 is a diagram illustrating a laser system 800 for use in a cell culture system in accordance with various implementations. The laser system 800 includes a laser source 802, along with a single-axis actuated mirror 804 electromechanically coupled to a motorized stage 806 and focusing optics 808, such that the mirror 804 and stage 806 are synchronized to produce a pattern of evenly spaced lines 810. The mirror 804 may be, for example, a galvanometer, microelectromechanical mirror, or spinning polygonal mirror. The mirror 804 is configured to move quickly enough to provide the desired point spacing given the fast PRR of the typical lasers (100+ kHz), while the coupled-stage motion may provide the slow desired interline spacing. The stage may be coupled to the sample with stationary optics or to the optics with a stationary sample.

There will be a small fixed angle between the perpendicular to the direction of travel and the scan line. For fixed scan rates, this can be accounted for in the relative orientation of the scan axis relative to the direction of travel. For variable rates of travel, this can be corrected by an optional second mirror 812 or through appropriate software processing of the desired resulting scan. This method can cover scans of an arbitrary size along one axis. Samples larger in the second axis may be scanned by tiling. The arrangement disclosed herein has a significant throughput advantage over the traditional tiled method of scanning as it eliminates the time necessary to step to a new tile. It also allows a wider field of view (FOV) to be scanned for a given optical system, as the tiled scan must be a rectangle inscribed inside of the circular FOV 814, while a single scan line can use the entire field diameter 816.

### In-Motion Adherent Cell Imaging and Laser Processing Platform

In optical engines for optical-based cell culture processing platforms, there is a need in the art to efficiently and stably couple laser energy into a moving optical platform when the power exceeds that which can be safely transmitted in a single-mode fiber.

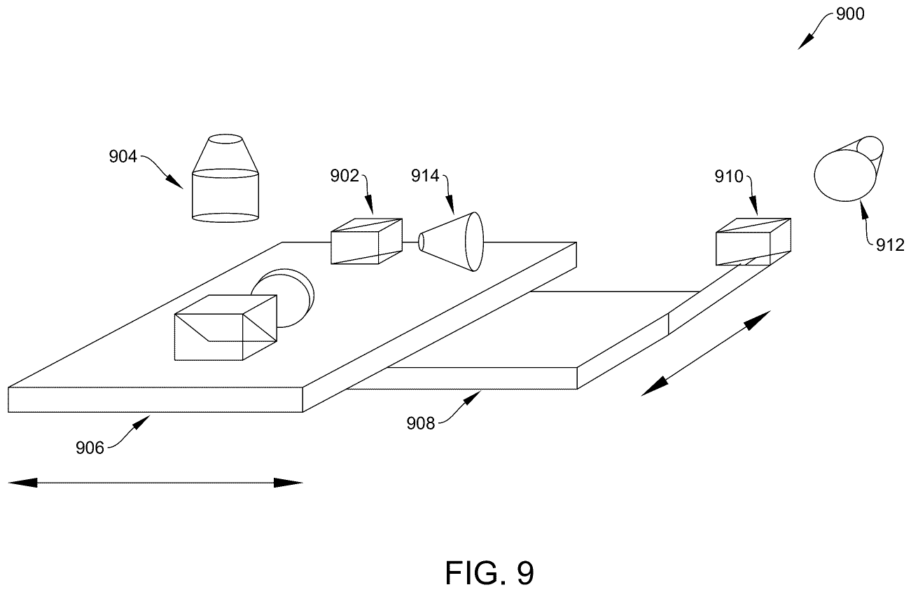

Systems and methods disclosed herein include an optical engine configured for in-motion adherent cell imaging and processing. FIG. 9 is a diagram illustrating a cell imaging and editing system 900 for use in a cell culture system in accordance with various implementations. System 900 includes a mirror coupled to an actuator 902 and appropriate scanning optics 904 placed on a first motorized linear stage 906, which is in turn placed on a second perpendicular motorized linear stage 908. This configuration allows for arbitrary travel in a two-dimensional plane. A mirror 910 is placed on only the second stage 908 and aligned to reflect a beam parallel to the axis of the travel of the second stage 908 to become parallel to the axis of travel of the first stage 906 and aligned with the optical system (i.e., actuator 902 and optics 904) present on the first stage 906. As all laser beams have a natural divergence, the size of the beam as it enters the optical system will depend on the position of the two stages 906, 908, with a difference in size equal to the beam divergence multiplied by the sum of the total travel of each stage individually. To keep the beam size within some uniformity tolerance, it may be advisable to add a beam expander 912 before the first mirror 910 and a beam reducer 914 in the beam path and attached to the first stage 906.

It will be appreciated that the motorized linear stages 906, 908 may be oriented in various configurations relative to a cell culture cassette in order to accommodate image capture in various X-Y-Z locations. For example, in some embodiments, linear stage 906 may be configured to move along a plane of the cell culture cassette. In some embodiments, linear stage 906 may be configured to move perpendicular to the plane of the cell culture cassette. In some embodiments, linear stage 906 may move in a first linear direction, and linear stage 908 may be mounted on linear stage 906 and configured to move in a second linear direction, such as perpendicular to the first linear direction.

Rapid Imaging with Continuous Autofocus

Rapid imaging performance increases sample throughput, but an accurate focal plane reference is necessary for correct data acquisition. In many instances, referencing a “global” surface topological model is sufficient, but localized variations in sample flatness may still impact model predictions. Thus, there is a need in the art for improved methods for correcting focus in rapid imaging systems.

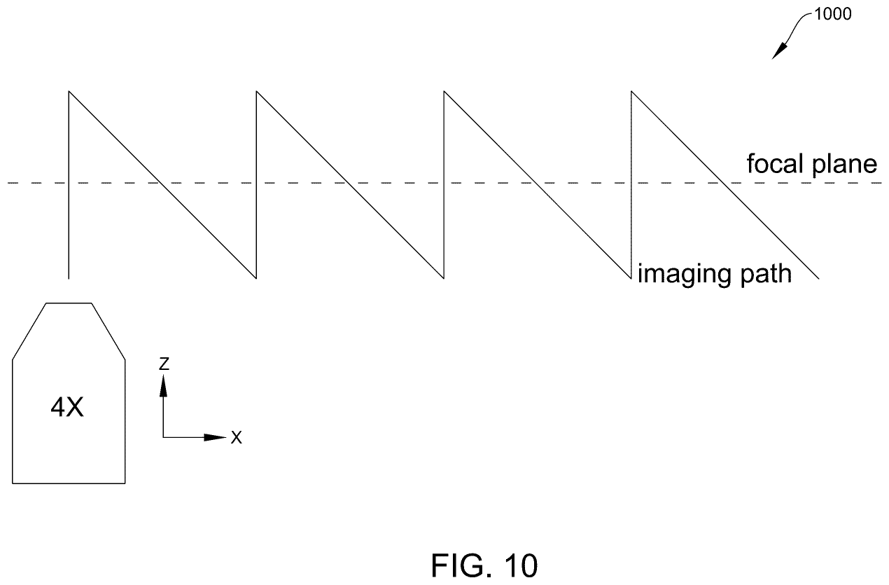

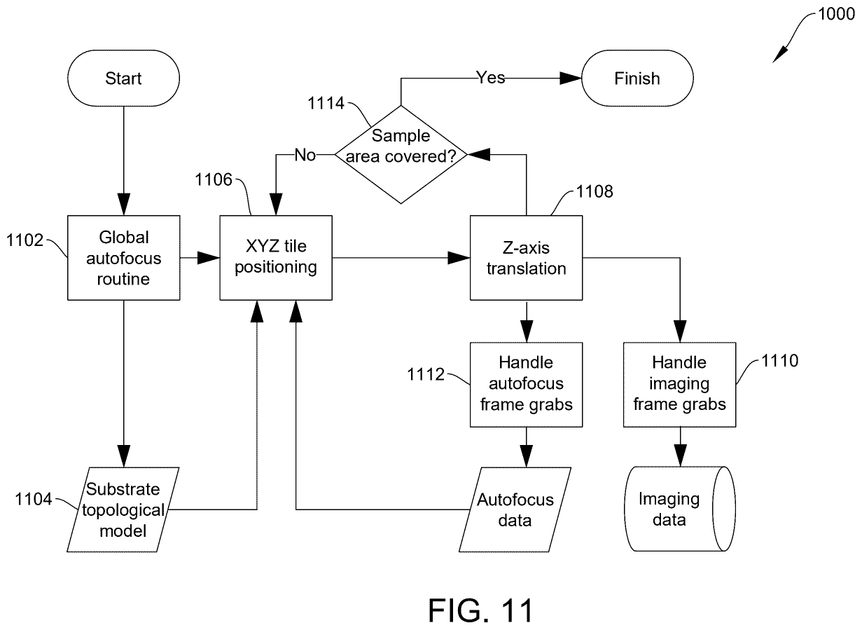

Systems and methods disclosed herein include rapid imaging systems that include continuous autofocus capabilities. FIG. 10 is a diagram illustrating an example of sawtooth imaging acquisition in accordance with various implementations. In the example shown in FIG. 10, autofocus may be computed in parallel and used to adjust the relative Z-position between subsequent XY steps. FIG. 11 is a flow chart of a method 1100 for performing continuous autofocus in a rapid imaging system in accordance with various implementations. In block 1102, the imaging system may perform a global autofocus routine. In block 1104, prior to imaging, the imaging system may compute a substrate topological model from focal samplings at the vertices of the predefined acquisition area. Often, these initial autofocus measurements are performed across a larger range to guarantee that the focal plane is captured. If the sample has large second-order variations in flatness (e.g., the substrate is not accurately modeled as a perfectly flat surface), then more points can be sampled across a mesh distribution to generate a closer approximation of the substrate topology. More specifically, the imaging system may be configured to measure various autofocus positions within the acquisition bounds, not just along the perimeter, to approximate a better model.

In block 1106, the imaging system is configured to position the imager in the X, Y, Z coordinates for image frame acquisition. In block 1108, the imaging system proceeds to capture Z-stacks along a serpentine path on the sample. While grabbing image frames in block 1110, the imaging system simultaneously calculates the focal plane position based on changes in a reflected laser spot geometry in block 1112. The newly computed focal position is used in a “feed-forward” mechanism to correct the Z-position in the adjacent imaging stack. In cases of autofocus failure, the system defaults to the Z-position interpolated from the global surface model. Performing image frame grabs and autofocus measurements simultaneously maximizes sample throughput while guaranteeing correct data acquisition. The imaging system determines whether the entire sample area has been imaged in block 1114, and continues image collection and autofocus measurement until the entire sample area has been scanned.

### Autonomous Slice Number Decision in Z-Stack Whole-Well Imaging

Whole-well imaging is time consuming, and iPSC colonies can exist at various thickness levels throughout a cell culture container. As a result, z-stack imaging is often applied to improve resolution when capturing images at low magnification (e.g., 4X objective). Since iPSC colonies can exist at various thicknesses, a fixed set of z slices may not provide an optimal resolution across the entire cell culture container. Increasing the number of z slices can significantly extend imaging time, while decreasing the number of z slices will compromise resolution, which likely negatively impacts accuracy of cell identification required for an autonomous laser colony management (LCM) process. Thus, there is a need in the art for improved methods of efficiently acquiring cell image data utilizing the optimal z-plane across the cell culture container.

Systems and methods disclosed herein include leveraging the autofocus to precisely define the top and bottom of each cell area at the cell level. Therefore, with a pre-defined cell area's thickness, an imaging system (e.g., the optical engine 104) may automatically determine the optimal number of z-slices required for each field. This approach enables an autonomous z-stack imaging method at the cell-container level that strikes a balance between imaging duration and resolution, ultimately providing the best results for accurate cell identification. As a result, this method enhances scanning settings and optimizes outcomes for the LCM process.

Following imaging, the imaging data may also be incorporated to drive autonomous laser settings (e.g., pitch, energy level, repeat scans) tailored to a specific cell area to improve scanning efficiency and minimize scanning duration. For example, the center of a colony is usually the thickest area, and cell identity is often not accurate due to high cell density. This leads to an inaccurate cell count, meaning the standard laser scanning parameters set for an entire cell culture container will likely not efficiently remove and kill cells. If autonomous image-based laser settings are incorporated, meaning the thickness of a specific cell area will automatically set the scanning parameters to a higher energy, a tighter pitch, and/or more repeats to remove and kill targeted cells more effectively. This helps avoid aggressive washing required for post-scan cell removal. Conversely, a cell colony's peripheral areas are usually the thinnest layer or most flat cell area, thus likely requiring more gentle laser scanning settings (e.g., lower energy, wider pitch, fewer repeated scans). Therefore, a similar autonomous image-based scanning setting approach may be applied to shorten scanning duration and minimize ECM destruction that later may benefit cell regrowth into the scanned areas when needed.

### Encapsulated Plasmonic Films for Cell Processing

With respect to laser films that enable optical imaging and manipulation in a closed cell culture container, there are a number of problems that must be solved. The problems include how to get a surface that is resilient and optimal for cell culture, minimize exposure to materials that may be desirable from an optical absorption/transmission perspective, and achieve absorption at longer wavelengths. For example, the fundamental absorption wavelength of gold nanoparticles is around 500-550 nm, but for some applications it would be desirable to use high-power lasers available in the near-infrared, specifically at 980 nm and 1064 nm. Thus, there is a need in the art for laser films that can enable optical cell culture processes but are resilient and do not impact the cell culture itself due to optical exposure.

Systems and methods disclosed herein include the use of pre-formulated nanoparticles available in multiple shapes, sizes, and materials, distributed onto a substrate, and then protected using a cell culture-compatible capping layer. One example of such a film would be gold nanorods, selected to absorb laser light at 980 nm or 1064 nm (but transmit well for imaging over most of the visible wavelength range) when encased in titanium dioxide that is deposited onto the surface of borosilicate glass, and then overcoated with a titanium dioxide cap layer which survives rapid heating from laser pulses absorbed by the nanoparticles. The film is configured to conduct heat to the cell media where it forms an explosive bubble that expands and collapses. The cap layer also resists erosion or degradation in cell media for long-term cell cultures. A similar film that absorbs at 532 nm may be constructed using gold nanospheres deposited or formed upon the surface.

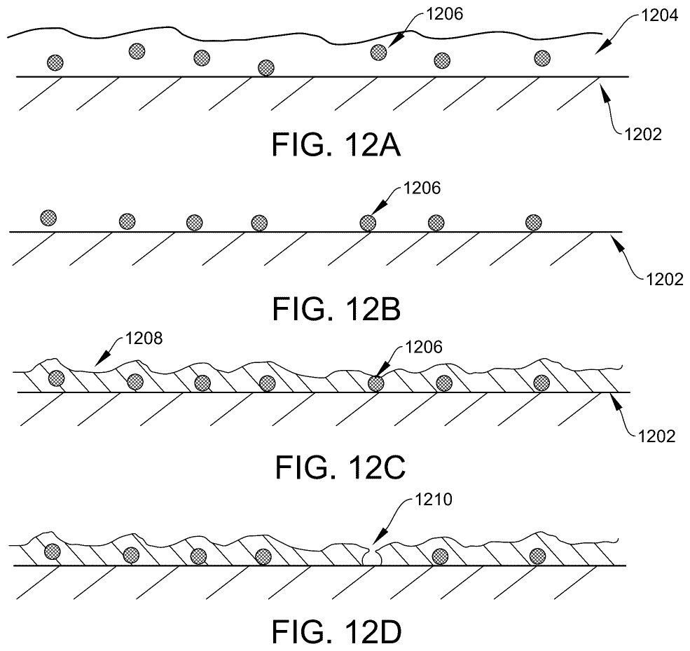

FIGS. 12A-D are diagrams illustrating a method of fabricating an encapsulated laser film in accordance with various implementations. FIG. 12A shows a transparent substrate 1202 (for example, borosilicate glass) with a liquid 1204 containing nanoparticles 1206. The nanoparticles 1206 may be nanospheres, nanorods, nanoshells, nanostars, or other synthesized nanoparticles. The nanoparticles 1206 may contain noble metals as the conductor (including but not limited to gold, silver, or platinum), or be made of compounds such as CdSe, CdTe, or InP. These nanoparticles may be pre-coated to resist agglomeration, to provide encapsulation, and/or to provide desirable surface properties for subsequent encapsulation. The uniform coating of the substrate 1202 may be accomplished by dip coating, spin coating, spray coating, Langmuir-Blodgett deposition, electrodeposition, or other means. In some implementations, the nanoparticles 1206 may be encased in a temporary shell, such as an organic compound, that prevents agglomeration and may set precise, controllable spacing between the nanoparticles 1206 on the substrate surface 1202. The temporary shell may be later removed by vaporization.

FIG. 12B shows the nanoparticles 1206 on the substrate surface 1202 after the containing liquid 1204 and any temporary shell material have been removed, typically by heating. An annealing step that processes the nanoparticles 1206 and/or embeds them into the substrate 1202 may be optionally performed at this point. FIG. 12C shows the subsequent coating of the nanoparticles 1206 and the substrate 1202 using a capping layer 1208. The capping layer 1208 is introduced to isolate biological processes performed on top of the capping layer 1208 from the nanoparticle material. The capping layer 1208 is also configured to conduct thermal energy from the nanoparticles 1206 into the cell media above it, without thermal damage. Such capping layers may be coated from materials including but not limited to titanium dioxide, silicon dioxide, silicon nitride, silicon carbide, titanium nitride, diamond-like carbon, and other biocompatible materials that withstand high temperatures as well as aggressive liquid environments. A final, optional step is shown in FIG. 12D. A selective etchant, which may be a wet or dry etchant, may be used to remove any nanoparticles 1206 that are exposed to the top surface (shown by hole 1210) to ensure no plasmonic material leaches into the cell media or cell product processed using the capping layer 1208. For example, when gold nanoparticles are used, an etch step with aqua regia may be used to remove any exposed nanoparticles.

### Substrate-Embedded Plasmonic Layers for Cell Processing

With respect to laser films that enable optical imaging and manipulation in a closed cell culture container, there are a number of problems that must be solved. Among the challenges is how to develop a cell culture surface that is resilient and optimal for cell culture while minimizing exposure to materials that may be desirable from an optical absorption/transmission perspective. Thus, there is a need in the art for laser films that can enable optical cell culture processes but are resilient and do not impact the cell culture itself.

Systems and methods disclosed herein include a range of semitransparent absorbing films that are embedded into a glass substrate, where they may absorb laser light and transmit thermal energy to the surface but not be exposed to the surface itself (so that they do not potentially perturb cell growth and health). The film includes nanoparticles/clusters/inclusions that are embedded in a glass that is suitable for cell culture, such as borosilicate glass. The absorbing material may be processed such that none of it is present on the top (cell culture) surface of the substrate. Additionally, the process may be controlled such that the majority of the absorbing material is less than 1000 nm from the top surface, and in some cases less than 500 nm, less than 250 nm, or less than 100 nm from the top surface, to efficiently transfer absorbed optical energy, as thermal energy, into the cell media.

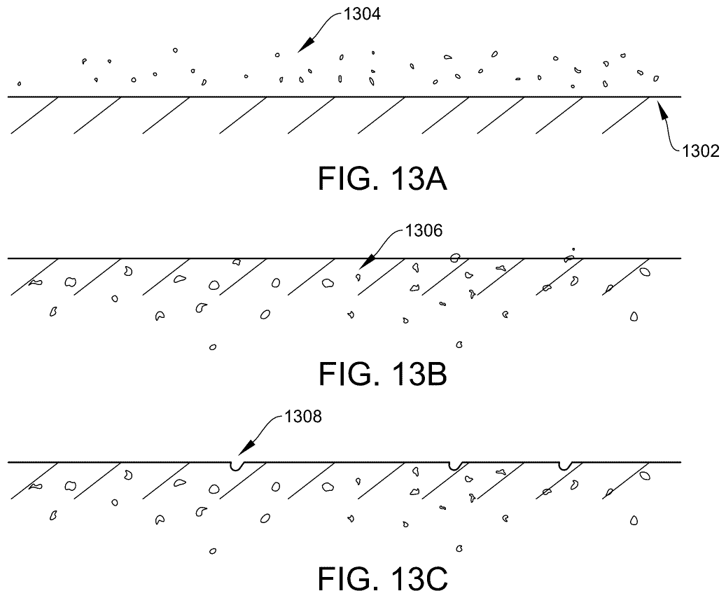

FIGS. 13A-C are diagrams illustrating a method for manufacturing substrate-embedded laser film in accordance with various implementations. FIG. 13A shows an absorber material 1304 that is disposed towards a substrate 1302 of a cell culture container. Methods of exposing the substrate include but are not limited to: ion exchange using molten salt baths, flame delivery (known as “fuming”), and/or ion beam implantation. The absorber materials 1304 may include, but are not limited to: gold, silver, platinum, copper, aluminum, CdSe, CdTe, or InP. The process, which may include elevated temperature, drives the absorber material 1304 into the substrate 1302. Subsequent annealing by oven or by optical means (e.g., laser, flash lamp) may be used to consolidate the optical material to nanoclusters and/or anneal the substrate structure, as shown by structure 1306 in FIG. 13B. A final optional step, shown in FIG. 13C, may involve selectively etching away any absorber material/nanoclusters that remain exposed to the surface, as indicated by holes 1308.

### Pre-Conditioning of Laser-Activated Cell Processing Films

Using semi-absorbing thin films for laser-based cell manipulation requires a material that can absorb a fraction of incoming pulsed laser light, thus producing heat which can be transferred into the surrounding medium, while also remaining sufficiently transparent to allow for brightfield imaging. To reliably operate a laser-based cell culture process over the course of several weeks, it is imperative that both the absorption and the transmission of this thin film remain constant throughout the subsequent laser treatments. Due to the substantial and rapid temperature increases and gradients that these materials undergo during a laser pulse event, it is common to encounter some degree of material phase change or breakdown which results in variability of local absorption and transmission, rendering the film no longer usable. Thus, there is a need in the art for more robust thin films for cell culture maintenance and manipulation in long-term cell cultures.

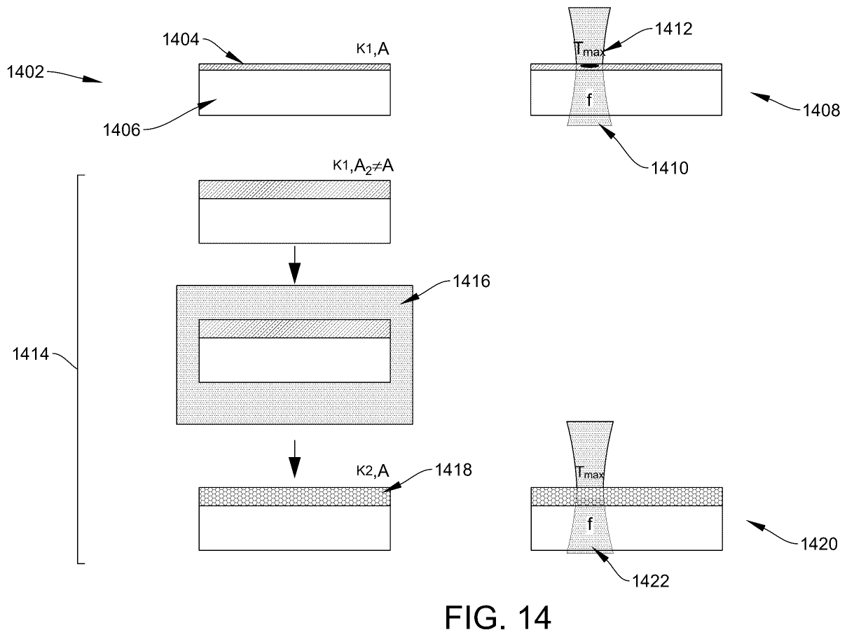

Systems and methods disclosed herein include thin film materials that are produced with the desired absorption at laser frequencies used for cell manipulation/removal. However, given the amorphous nature of the deposition process, the films may be prone to some degree of crystal formation when laser treated and heated above some critical temperature. FIG. 14 illustrates a method for pre-conditioning of a laser film for cell culture process applications in accordance with various implementations. In step 1402, a thin absorbing film 1404, with absorption A and extinction coefficient κ1, has been deposited onto a transparent substrate 1406. In step 1408, an incoming pulsed laser 1410 with fluence f heats the absorbing film 1404 up to some operational temperature for cell removal, and produces a maximum temperature of Tₘₐₓ within the absorbing film 1404. Due to the unstable nature of the amorphous film, a region 1412 of visible change occurs, altering the absorption and/or transmission in that region.

To counteract this structural change that occurs during operation, a thermal annealing process 1416 is applied to the laser film in step 1414. During the annealing process 1416, some amount of controlled phase change occurs, which results in an increased operating temperature range for laser-based cell removal while still preserving sufficient absorption. To adjust for any absorption changes which may occur during the annealing process 1416, the initial thickness of the deposited layer may be different than the un-annealed case, resulting in a different absorption. This thickness could be chosen such that after the annealing process 1416, the resulting film 1418 would have the same desired overall absorption, although the extinction coefficient may be different. In step 1420, subjecting the treated film 1418 to a laser pulse 1422 would produce the same (or similar) Tₘₐₓ on the top surface while withstanding any local changes in absorption or transmission. In some implementations, the absorbing film may be metallic or dielectric, and may be a single layer or include multiple layers of different materials. Deposition methods may include any common thin deposition techniques such as evaporation, sputtering, or chemical vapor deposition. Thermal annealing may be done isothermally using an oven or furnace, or from one surface only, such as by using a flash lamp or laser. In some implementations, atmospheric environmental parameters (e.g., gas mix) may also be controlled while performing temperature conditioning.

### Glass-Embedded Plasmonic Films for Cell Processing

With respect to laser films that enable optical imaging and manipulation in a closed cell culture container, there are a number of problems that must be solved. Among the challenges is how to develop a cell culture surface that is resilient and optimal for cell culture while minimizing exposure to materials that may be desirable from an optical absorption/transmission perspective. Thus, there is a need in the art for laser films that can enable optical cell culture processes but are resilient and do not impact the cell culture itself.

Systems and methods disclosed herein include a film that is disposed on a transparent, biocompatible surface such as borosilicate glass, that provides a resonant plasmonic absorption function enabling a semi-transparent absorbing/reflecting layer. This film includes nanoparticles of a noble metal that are partially embedded in the glass, such that they withstand significant forces (including shear forces from liquid operations as well as mechanical surface handling, forces from explosive microbubble expansion and collapse, forces from rapid heating and cooling) without ablation of the nanoparticles into the liquid that they contact. The film may be used for laser processing of cells and/or extracellular matrices or other coatings. The cell processing may include, but not be limited to, intracellular delivery or cell removal. The mechanism for such cell and ECM processing may be thermal, microbubble explosion and collapse, or both. For such processes, either continuous-wave (CW) or pulsed laser sources may be used, or in some cases LED sources. The plasmonic layer may further be used for sensing applications, including but not limited to sensing the liquid, ECM and/or cell environment by resonant wavelength shifts (as measured by reflection, transmission, and/or absorption spectra), surface plasmon resonance (SPR) sensing, or surface-enhanced Raman spectroscopy (SERS).

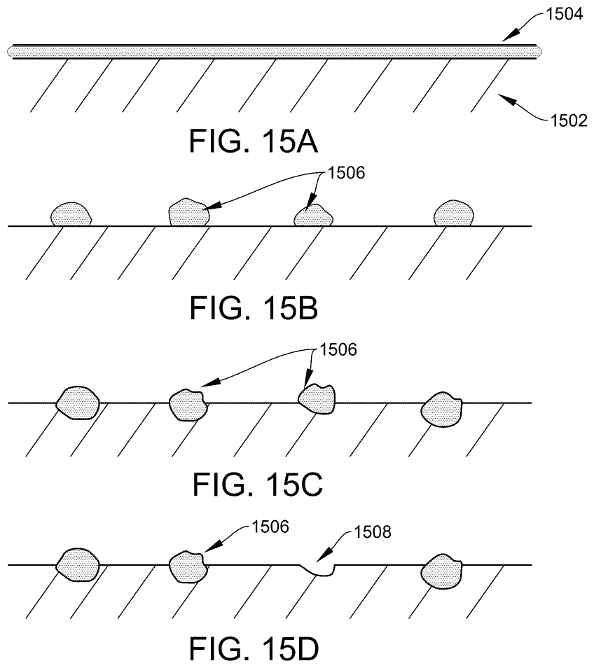

FIGS. 15A-D illustrate a method for manufacturing a plasmonic film suitable for long-term cell culture in accordance with various implementations. FIG. 15A shows an optional step in which a source film 1504 is deposited on a clear substrate 1502. The source film 1504 may, for example, include gold with a thickness of a few nanometers. It may be annealed as part of the overall method to form nanoparticle islands 1506 as shown in FIG. 15B, which will become plasmonic resonant islands. In alternate implementations, the source film 1504 may be deposited under conditions that result in nucleation and growth of the islands 1506 during deposition itself, including but not limited to: pretreatment of substrate surface to minimize wetting of the plasmonic material to the surface; pre-coating of surface with a thin temporary layer that minimizes wetting; pre-roughening of the substrate surface by ion beam, etching, or other means; deposition at low temperatures (such as lower than 200° C.); and/or rapid deposition to minimize formation of contiguous films by material migration. FIG. 15B shows individual nanoparticle islands 1506 on the substrate surface. These may be formed from a deposition as described herein, potentially with some subsequent annealing to consolidate islands, or the nanoparticles may be directly deposited on the surface using a number of other techniques described herein.

FIG. 15C shows the nanoparticle islands 1506 after embedding into the glass surface by high temperature treatment (“annealing”). The substrate 1502 may be laid horizontal for the annealing process, on a flat surface, so as to minimize warping from gravitational effects as the glass approaches its softening temperature. Further, temperature ramp-up and ramp-down may be done slowly to minimize any thermal gradients that may cause substrate warping. The surface under the substrate 1502 is preferably a high melting point, high conductivity material such as silicon or alumina. After embedding of the nanoparticles, a challenge or cleaning procedure may be applied as shown in FIG. 15D. This procedure mechanically challenges the surface such that any non-attached or loosely embedded nanoparticles are detached and removed as indicated by hole 1508, whereas well-attached nanoparticles 1506 remain. The procedure may include, but is not limited to, washing with high shear forces, ultrasonic baths, pulsed laser treatments, surface wiping with clean materials, and/or tape adhesion and removal. The surface treatment may be measured by optical property changes.

The surface may be manufactured to match a target resonant wavelength. For example, very small nanoparticles of gold and/or silver, either pre-formed and applied to the surface, or formed from a source film, may be used to match a 532 nm laser wavelength. In other applications, the embedded particles may originate from gold nanorods and have resonant absorption bands covering the 980 nm and/or 1064 nm laser bands.

Compensation for Discoloration from Sterilization

Laser-based cell manipulation of a cell culture chamber depends on well-controlled laser pulses delivered to the cell culture surface. Sterilization methods such as gamma irradiation and e-beam irradiation discolor the glass substrate of the surface, causing the glass to attenuate the laser light on its way to the cell surface. Run-to-run variations in sterilization dose will lead to batch-to-batch variations in laser attenuation. Thus, there is a need in the art for consistent sterilization methods of cell culture surfaces that do not interfere with optical cell processing mechanisms.

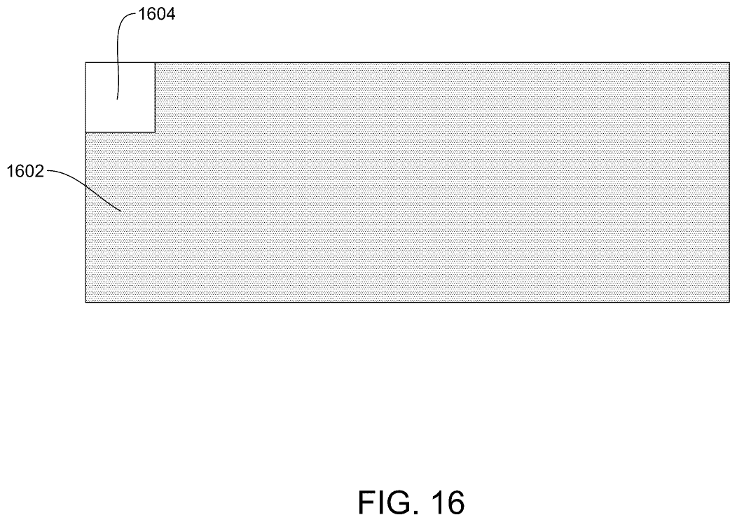

Systems and methods disclosed herein include methods for compensating for discoloration when sterilizing a glass substrate used as a cell culture growth surface. FIG. 16 is a diagram illustrating a method of constructing a cell culture surface in accordance with various implementations. In FIG. 16, a section of glass substrate 1602 may be masked during film deposition, leaving a section 1604 of the glass uncoated. A window in the cassette assembly may be placed such that the uncoated section 1604 is optically accessible after assembly. When the cassette assembly is sterilized, the glass substrate 1602 will discolor, increasing the light attenuation. The attenuation of light through the window (and as incident in section 1604) may be measured at the laser operation wavelength. The laser operation energy may be scaled accordingly to compensate for the change in attenuation caused by sterilization (e.g., the greater the attenuation, the higher the laser operation energy should be).

In an alternate implementation, an uncoated reference glass may be included in each sterilization run. The attenuation of light through the reference glass may be measured after sterilization. The sterilization run number associated with each cassette may be tracked, and the laser operation energy for each cassette may be scaled according to the reference glass from its sterilization run. In another alternative implementation, the laser film may completely coat the glass, but a window in the cassette is left so that the net attenuation of film and glass may be measured by the optical engine. An example of doing this would be the region of the fiducial marks. The net amount of light that passes through may give an indication of the net attenuation presented by gamma-irradiated glass plus the film, from which factors to correct laser power may be determined.

### Closed-Loop Biological Experiment Monitoring and Support

When monitoring cell culture processes, it is the responsibility of the operator to manually monitor a biological experiment in a cell culture container such as a chamber, well plate, or other container to identify any issues that negatively impact outcome. The larger the experiment or set of experiments, the higher the monitoring burden. To avoid unpredictable failures and plan interventions, the operator must manually inspect and characterize the experiment on a regular basis. Thus, there is a need in the art to automate aspects of cell culture processing monitoring.

Systems and methods disclosed herein include a closed-loop system that ties experiment grading to the delivery of technical support to provide ongoing experimental status along with recommendations to the experiment operator. This closed-loop monitoring system operates on a system that includes a cell culture chamber for biological experiments. The cell culture chamber may enable various functions, such as fluidic operations, imaging, and the application of a laser or other energy source. The cell culture chamber may be monitored by multiple sensors, including video, temperature, and others.

Images of the contents of the chamber-based system may be collected on a regular basis, along with sensor data. After each image acquisition of a cell culture chamber, the closed-loop monitoring system may be configured to assign a grade to the chamber indicating the condition of the chamber. The grade of the condition may be on a scale (e.g., color-based, numerical range) indicating if the experiment is progressing as expected or is demonstrating issues requiring operator intervention. The grade may be composed of multiple inputs, including but not limited to cell culture images; detection of cell colonies, debris, and anomalies and other morphological inputs; estimation of cell colony health; sensors internal to the chamber (e.g., temperature, pressure, liquid levels); sensors external to the chamber (e.g., temperature); and observations input by the operator. The grade, along with the point in time when it is created, may indicate chamber status. A timeline of chamber status may be kept throughout the duration of the cell culture process.

The closed-loop support system may be configured to guide the operator when the chamber status moves out of the acceptable status at any point in time. Acceptable and non-acceptable statuses and corresponding grades may be predefined. The guidance may assist the user to determine next steps in improving the cell culture status or to terminate the process. This guidance can come in a variety of forms, including but not limited to intervention or support from an expert operator or scientist (either in person or virtually), automatic delivery of support documents, and automatic delivery of specific guidance, including adjustment of equipment or cell culture parameters.

After operator intervention is complete, the closed-loop system will update the status and ask the operator to confirm the status based on their own observations. A centralized dashboard may show experiment status as a timeline for all cell cultures in process and all historical processes. This dashboard may be configured to show changes in status, percentage of processes recovered, success of automatic and manual interventions, and agreement between automatic and manual status inputs.

### In-Process Manufacturing Change Based on Biological Observations

Each cell line has differing characteristics as it relates to case of manipulation in automated manufacturing when cultured in a cell culture container. While some cell lines may be relatively easy to manipulate and have a high likelihood of producing cells that pass all QC tests at the end of a process, others may be more difficult. To accommodate the more difficult cell lines, if each cell line is treated the same, the process will have to be scaled up, leading to overproduction and waste for some more efficient cell lines. Thus, there is a need in the art for dynamic manufacturing changes in automated cell culture processes.

Systems and methods disclosed herein include a system (e.g., a computing subsystem) configured to monitor the biological process in each stage of a manufacturing process and configured to adjust downstream production based on those observations. These observations may be made over a single, or series of images, as well as video, temperature, environmental measures, internal sensors, and other data sources. The system may base downstream production changes on prior manufacturing runs to make these assessments and take into account supplies and capacity of the on-premise system. The system may be configured to make adjustments to a variety of parameters based on the observations, the parameters including but not limited to number of cell culture containers, seeding density, energy level for removal, scanning cadence, length of reprogramming phase, length of stabilization phase, length of expansion phase, and signaling factors timing and dosage.

The system may be configured to provide the operator with an opportunity to override each of these parameters. For one-time decisions, such as seeding density, the system may be configured to prompt the operator to confirm the choices and ensure enough consumables are available for the process. After operator intervention is complete, the system may update and run with the chosen parameters.

### System for In Situ Laser Effect Monitoring

The present implementations disclosed herein include systems for using pulsed lasers and an absorbing layer disposed on the interior of a cell culture container, for processing cells using laser-initiated bubbles. The mechanical forces imparted upon cells, and therefore the effect (whether the goal is temporary membrane poration for intracellular delivery, or cell death), is dependent on the energy transfer from the laser beam to the absorptive film, and then to the liquid media where a bubble may or may not initiate, grow, and collapse. As a result of this chain, there is a complex set of dependencies including laser pulse power, laser spot size, laser polarization, laser pulse length, laser incidence angle, reflections or absorption from an underlying substrate or other intervening materials, absorption in the laser absorbing film (including the spatial/axial distribution of absorbed energy within a film) which may have substrate-to-substrate or intra-substrate variations, conduction of absorbed energy to the surface of the laser film where it makes contact with cell media, the surface conditions of the film including roughness, ECM or other biofilm coatings, and the liquid (cell media) conditions including but not limited to temperature and dissolved gas concentrations. In short, simple monitoring and closed-loop control of laser pulse energy may not provide a sufficient picture of the ultimate mechanical effect on target cells. Thus, there is a need in the art for improved methods of monitoring optical-based cell manipulation techniques.

Systems and methods disclosed herein include using at least one scattered light detector to detect light being scattered from laser-initiated bubbles in an optical-based cell manipulation approach. In some implementations, a light source other than the bubble-activating (“excitation”) pulsed laser is used. For example, a continuous wave light source at a different wavelength than the excitation laser may be used to enable wavelength filtering prior to detection, and continuous measurement of scattering prior to, during, and after the excitation laser pulse. In other implementations, the excitation laser may also be used as the bubble profiling laser, so as to minimize the hardware overhead required. This is possible if the pulse rate of the laser is fast relative to the targeted bubble lifetime. For example, if a 500 kHz pulsed laser is used and the target bubble lifetime is 20 microseconds, roughly 10 measurements of the bubble may be made, either in a single shot, or at one timepoint per bubble excitation pulse.

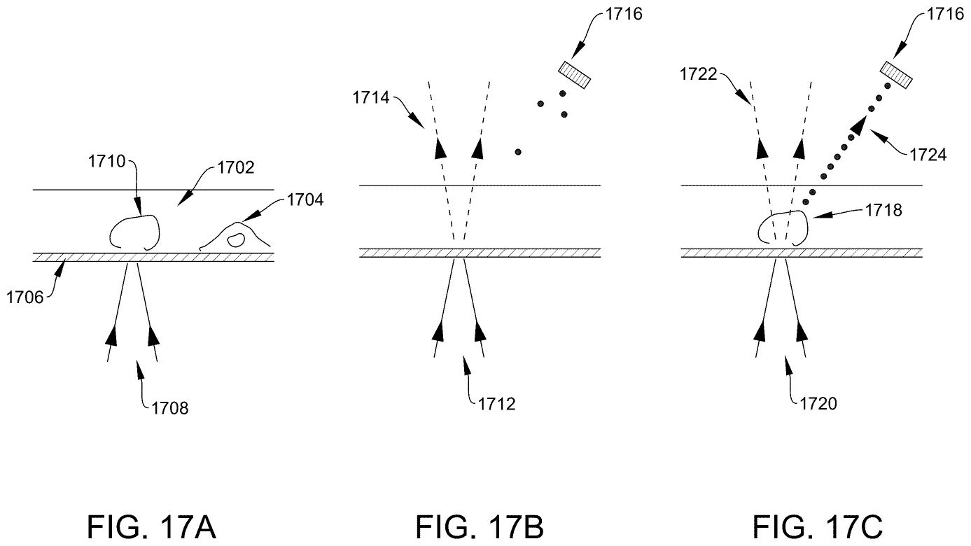

FIGS. 17A-C are diagrams illustrating methods for in situ laser monitoring in a cell culture system in accordance with various implementations. FIG. 17A shows a general representation of an optical-based cell culture system with a cell media volume 1702 and cells 1704 growing on a surface that includes a laser-absorbing film 1706. The film 1706 is configured to absorb incoming laser pulses 1708 and triggers the formation, expansion, and subsequent collapse of microbubbles 1710. The size and duration of the bubbles 1710 are important for causing different effects on the cells and/or ECM disposed on the cell culture surface. FIG. 17B shows a low energy (“probe”) laser pulse 1712 that does not trigger a bubble. Because the laser films in use are partially-absorptive, a fraction of the laser light passes through the film and media as indicated by beam 1714. A scattered light detector 1716 is placed off-axis where directly-transmitted laser light is not directed. In some implementations, multiple off-axis detectors may be used. The off-axis detector detects any low level of scattered laser light in the system, which serves as a baseline signal.

FIG. 17C shows the same setup as FIG. 17B, except that a bubble 1718 has been initiated by an excitation pulse. A probe pulse 1720 is then used to illuminate the bubble region. Much of the light still passes through as indicated by beam 1712, but because of the shape of the bubble volume, some light also scatters off-axis as indicated by beam 1724 and strikes the scattering detector 1716. The difference between the previously-measured baseline and probe signal after bubble initiation is an indication of bubble presence, size, and/or shape. This shape and size measurement may be correlated on a separate specialized setup with strobe images of the bubble, or be directly previously correlated with biological cell effects.

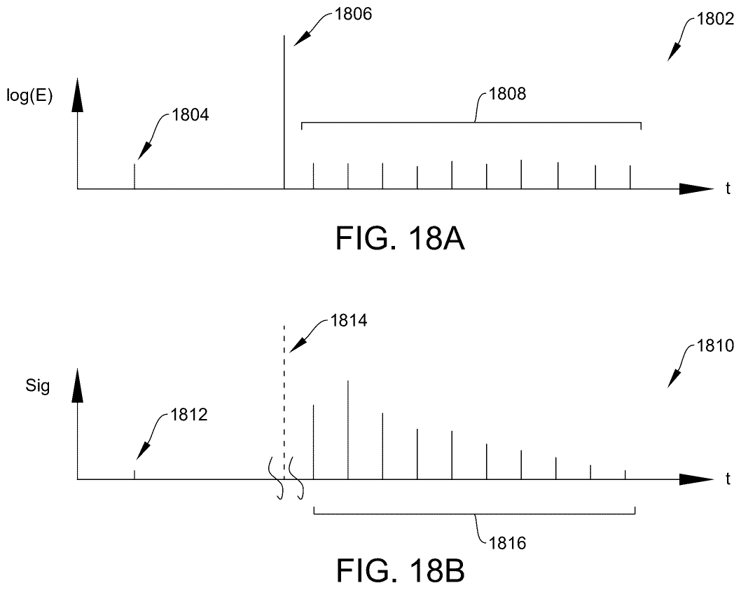

FIGS. 18A-B are plots showing laser pulse and scattering detector signals in accordance with various implementations. FIG. 18A shows laser pulse energy transmitted to the sample, in which a single laser is used for both excitation and probe pulse sequence 1802. In the example implementation shown in FIG. 18A, a pulsed laser is used in conjunction with an acousto-optic or other modulator that provides rapid (single pulse) energy control. In this example, the laser is pulsed at 500 kHz (2 microsecond intervals), although such a system could use pulses into the 1 MHz range or faster, or 100 kHz or less. In this example, the bubble duration is roughly 20 microseconds. A reference probe pulse 1804 is used to measure baseline light scattering without a bubble present. Then an excitation pulse 1806, which is significantly higher than probe pulse 1804 and sufficient to trigger bubble initiation and expansion, is transmitted. Then, a train of probe pulses 1808 may be used to illuminate the bubble region. In other implementations, a single probe pulse at a set time after the excitation pulse 1806 may be used, potentially to simplify the detection electronics, or minimize the potential interaction of the probe pulses with the laser film or bubble.

FIG. 18B shows a corresponding signal trace 1810 on the scattering detector. A baseline light scattering level 1812 is measured from the initial probe pulse, which corresponds to light scattered by elements other than the bubble. Scattering from the excitation pulse 1814 is removed from the plot. Then scattered light signals from the train of probe pulses 1816 are recorded, with the level over the baseline indicative of bubble presence, size, and shape. This measurement may be repeated on the same film position to measure the effects of multiple excitation pulses on the same region of film, ECM, and media volume. This measurement may also be repeated at multiple positions within a cell culture container to measure spatially dependent variations in bubble effect. The measurement may be performed at the first use of a cell culture container, or before every laser cell operation in a cell culture container, to account for variation in surface and media conditions. The measurement may be made for a series of excitation pulse energies to map bubble threshold and energy transfer parameters.

### Closed-Loop Laser Cell Treatment Measurement

When laser-treating cells for either temporary poration (to facilitate intracellular delivery or extraction) or to terminate them, the energy level required to achieve the desired effect may vary significantly from cell to cell, batch to batch, day to day depending on cell state (level of adherence, density, gene expression, size/volume, etc.), media composition and state, and many equipment- and consumable-related factors. Moreover, the lag between application of laser energy (either directly to the cell, to the cell medium, or to an absorbing element disposed near the cell) to thermally or mechanically disrupt the cell and receiving evidence of the ultimate intended effect (for example, change in gene expression due to successful intracellular delivery, or death of the cell as evidenced by its detachment from a surface) can be lengthy. A better method by which laser-generated thermal, chemical, or mechanical effects on cells can be measured with a faster feedback loop, and with higher spatial resolution, is needed.

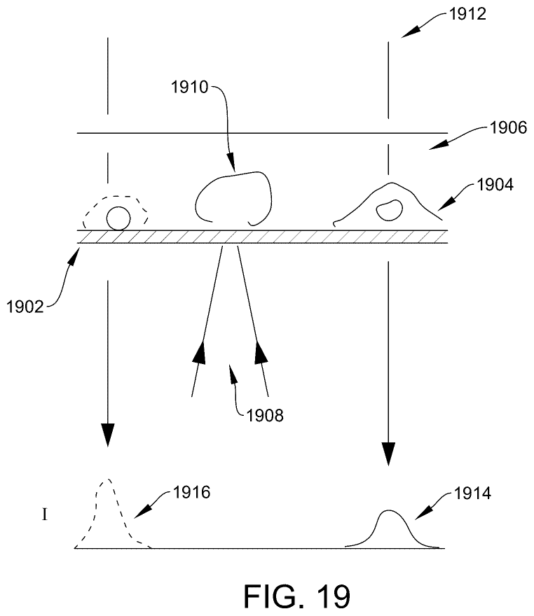

Systems and methods disclosed herein include using transmitted-light imaging of a cell culture prior to and after laser treatment to assess effects on cells with rapid feedback potential. FIG. 19 is a diagram illustrating application of a laser pulse on a cell culture surface in accordance with various implementations. A laser-absorbing film 1902 is located on a surface of a consumable that contains cells 1904 and media 1906. The film 1902 is configured to absorb laser light pulses 1908 and trigger rapid expansion and collapse of microbubbles 1910 that impart mechanical forces on cells. These mechanical forces may be used to temporarily porate the outer cell membrane for intracellular delivery or sampling operations. In other applications, it may be used to impart sufficient forces on the cell membrane, cytoskeleton, focal adhesions, or other components to cause cell death. In many cases, it is desirable to use the minimum amount of laser pulse energy required to achieve the intended effect to minimize collateral cell damage, overall cell media heating, and damage to the laser-absorbing film.

The present implementations use a transmission illumination system 1912 to image the cells. Images may be acquired at a single Z plane that provides contrast on key cellular components, for example at a plane where the cell nucleus is shown with good contrast. The contrast for any object, whether it be cell clusters, cell body, nucleus, nucleoli, or other cell components, depends on the relative refractive indices compared to surroundings. In this example, an image profile 1914 before laser scanning shows a signal from the cell nucleus when surrounded by cytoplasm within an intact membrane. After laser treatment, the outer cell membrane has been porated, causing a drop in the refractive index of the cytoplasm (which may be enhanced by controlling osmolarity of the cell media), and imaging the same cell shows a higher contrast image 1916 of the nucleus. Additionally, there may be spatial shift or size changes in the cell, or there may be blebbing or other indications of cellular damage and/or self-repair mechanisms. The example shown in FIG. 19 focuses on nuclear contrast, but other cell components or cell cluster components may be imaged and compared by similar means. In some implementations, phase contrast imaging or quantitative phase imaging may be used.

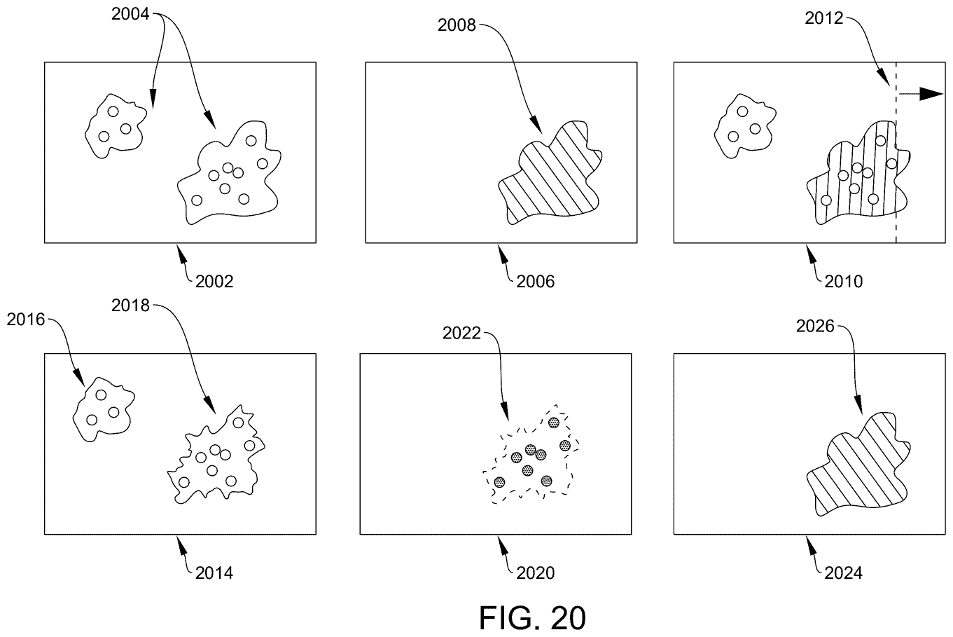

FIG. 20 are images showing laser scanning of cell colonies in accordance with various implementations. Image 2002 shows an initial image of a cell culture 2004 with two cell colonies. Image 2006 shows a cell scanning map that has been requested, where one of the two colonies will be laser-treated as indicated by shaded region 2008. Image 2010 shows a laser scan (in this example, a raster scan 2012 traversing the field of view) performed on the cell culture. Image 2014 shows another transmission image after scanning, in which an unscanned colony 2016 has a substantially unchanged image but a scanned colony 2018 shows differences (sometimes subtle) in its image. Image 2020 shows a differential image in which pre- and post-scan images have been compared, enhancing the contrast of laser-induced changes on scanned cell colony 2022. Finally, image 2024 shows a processed version of the differential image in which regional changes 2026 due to laser scanning may be mapped and confirmed against the scan plan.

The combination of imaging and scanning may be done in a variety of approaches. In one implementation, the entire cell culture is imaged, then the entire cell culture is laser scanned where desired, then another complete image is acquired. In another implementation, the pre-imaging, scanning, and post-imaging may be done on a field-of-view by field-of-view basis, when laser scanning and imaging are performed on multiple fields of view per cell culture. In other implementations, in which a continuous-motion scanning system is used, a sliding field of view may be imaged, with the “pre-scan” images taken on the leading edge, and “post-scan” images on the trailing edge of the field of view. The laser scan may occur along a line perpendicular to the continuous field of view motion, between the pre- and post-scan imaging regions of interest at the leading and trailing edges, respectively.

Information from the differential laser effect imaging may be used in a number of ways in the cell culture system, including but not limited to the following, and combinations of the following: (1) immediate feedback to the laser scanning subsystem/routines, whereby cells or regions of cells may be re-scanned to impart laser effect, or whereby the energy of laser pulses is modulated in order to produce the desired effect level as reflected by differential imaging; (2) profiling laser effects across a population of cell types, states, configurations, etc. and/or a range of laser processing parameters or other cell culture conditions, to assess thresholds, ranges, variability of laser effects as a function of cell culture state; (3) feedback to cell culture tracking and management systems regarding laser effects on cells. The feedback may change laser parameters over longer periods of time. The effect level on cells may be indicative of cell state, which is mapped and tracked in a cell culture management system. Real-time observations of the process by which cell colonies retract or develop high contrast indicate that the optical alterations typically develop over the course of 1-60 seconds. These subtle optical changes allow workers to confirm that adequate laser treatment has taken place and that the well plate or cassette may be removed from the laser instrument. The 1-60 second timescale may be far longer than the time needed to scan/zap and image a single field of view. To most rapidly scan/zap cells, it may be most efficient to scan/zap ˜100 different fields of view over the course of 60 seconds, and then cycle back to the first field of view to image the colonies so as to ascertain whether they are sufficiently laser treated.

### Systems and Methods for Multi-Imager Registration for Model Training

Ultra-high throughput imaging for cell cultures is required to scale cell manufacturing. Label-free imaging such as transmission imaging is strongly preferred because of throughput requirements and labeling restrictions for clinical cells. Specialized imaging systems to perform this imaging are built, typically prioritizing throughput over spatial resolution. However, to process the enormous amount of cell image data into maps that can be used for decision making (such as cell confluence, cell density, cell phenotype, cell dynamics such as proliferation and motility), deep learning models are often employed because they generalize well from training data into new situations. To generate training data, ground truth datasets must be available in addition to the label-free images from the imaging subsystem. This may require substantially different imaging systems from typical high-throughput imaging equipment. Temporal and spatial coordination between imaging equipment and image streams must also be solved. Thus, there is a need in the art for high throughput imaging systems compatible with machine learning methods.

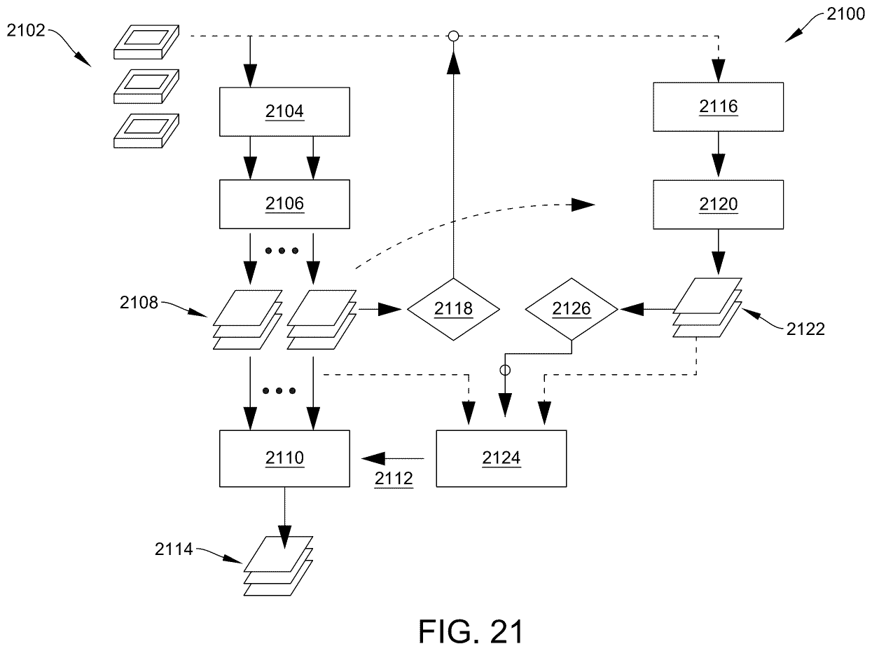

Systems and methods disclosed herein combine high-throughput label-free imaging systems with high-content imaging systems through the use of co-registration between the two systems. Multiple methods of co-registration are described herein. Moreover, selective use of high-content imaging paired with high-throughput imaging is described herein. FIG. 21 is a diagram illustrating an imaging and image processing system 2100 for use in a cell culture system in accordance with various implementations. The system 2100 includes a series of consumables 2102 that is imaged by a high-throughput live cell imager 2104. The imager 2104 is configured for high throughput and minimum imaging times, for time series imaging of cell culture processes in the consumables 2102. The consumables 2102 may be, for example, well plates, flasks, and/or fluidic cassettes, including fluidic cassettes with aseptic connections. Multiple time points may be imaged of a particular consumable.

An image pre-processing system 2106 provides imager-specific image corrections, including but not limited to X, Y, and Z registration and calibration (including with autofocus and registration methods discussed herein), digital refocusing, image de-warping, illumination correction, computational phase calculation, and/or image stitching, to produce output that is the aligned/corrected “runtime” images at multiple timepoints. As shown in FIG. 21, a stack of images 2108 indicates multiple Z planes, or multiple outputs from a computational phase technique, such as phase delay, relative intensity, total Z depth. The X, Y registration may be to a universal coordinate system that is valid across imagers, and imagers of different types, to allow registration of labeled images with label-free images. Multiple stacks of images indicate multiple time points at the same spatial location. These images are in turn provided to a computing subsystem 2110, which may include artificial intelligence models such as deep learning models that use parameters (weights) 2112 that have been optimized based on previous training data. The output of these models is one or more maps 2114 of the cell culture that may include but not be limited to maps of: cell confluence, cell density, cell phenotype, cell growth rate, cell layer height, debris, cell attachment, etc.

Training of the deep learning models to map cells from high-throughput label-free modalities typically requires the capture of additional, labeled images in parallel with the label-free images. This may be done on a different imaging system 2116, in which a subset of cell cultures, potentially at a subset of time points, are routed to this second type of imaging system. In some implementations, a decision system 2118 may be used to make decisions on whether to route cell cultures/consumables to the second imager 2116. For ML training, it is important to create well-balanced training datasets which cover modes of observed patterns in a given domain of images well. The decision system 2118 may be configured to actively assess the input image and the overlap of its regions of interest (ROIs) with the modes of training dataset distribution. If an out-of-distribution ROI is observed or if an ROI from an under-represented mode is observed, then the decision system 2118 may select this input image for secondary imaging of labels for it. Such a selective labeled image acquisition strategy would optimally increase the paired ground truth dataset size and prevent unnecessary labeled data acquisitions from well-covered modes.

In some implementations, paired labeled data acquisition decisions may be made based on an embedding model which processes each label-free image patch/field/composite to calculate embedding vectors that are descriptive of image patterns observed in the data domain. During model training phases, such embedding vector spaces can be constructed and significant modes of this space can be identified. These modes and coverage from within these modes would be input to the decision system to identify regions of interest (ROIs) in the label-free image based on how well they extend the previously-sampled space of label-free images for which a labeled image exists in a training dataset. The goal of such a decision algorithm would be to create an even distribution across the population of images as mapped onto the embedding space.

In other implementations, “active learning” may be used to select ROIs with embedding vectors matching regions where model learning can most benefit (e.g., where label-free-to-map model performance is suboptimal). For this purpose, regions in the embedding space where the models generate wrong or inconsistent predictions need to be identified. Common techniques for such identification include using multiple versions of the same model and using differences between their prediction maps as inconsistent firings, or using false alarms and misses of a given model as compared to labeled ground-truth, or using manual annotations to identify false alarms/misses of a given model. Given such identified ROIs where models fire inaccurately or inconsistently, then the embedding space distributions of these ROIs can be used to select ‘more’ ROIs that are similar to them in the new label-free images. In other implementations, decisions to select cell cultures for labeled imaging may be done manually.

The decision system 2118 to select cell cultures for labeled imaging may also operate in an online manner and continue to run during cell manufacturing processes. If the ground-truth process is sacrificial, then this system may actively select cultures for further labeled imaging in only the situations where the cell culture would be lost anyway, for example to an uncontrolled spontaneous differentiation event. Cell cultures with designated ROIs may be imaged in their entirety. In other cases, only regions corresponding to the ROIs may be imaged. This is particularly helpful if the labeled imaging process is low-throughput (for example, if it is high-magnification, involves many Z slices, uses faint fluorophores, or uses techniques such as Raman spectroscopy which involve long exposure times).

Labeled images acquired on imaging system 2116 may include but not be limited to fluorescent widefield microscopy, point scanning confocal microscopy, spinning disk confocal microscopy, 2-photon microscopy, light sheet microscopy, total internal reflection microscopy, and/or super resolution microscopy. Fluorescent elements may include but not be limited to organic dyes, protein-based fluorophores, quantum dots, fluorescent nucleotides, fluorescent proteins, small molecule fluorophores, near-infrared dyes, bioluminescent markers, and/or fluorescent nanoparticles. Fluorescent proteins may include proteins expressed by the cells themselves, sometimes as a measure of gene expression. Fluorescence in situ hybridization (FISH) and RNA-FISH, as well as multiplexed FISH, may also be used to image the cell culture. Imaging may involve terminal processes such as fixing and staining, or may be performed on live cells, with the consumable returned to incubation after imaging.

The images collected by imager 2116 are corrected and aligned in a second computing subsystem 2120. The alignment may include alignment in X, Y to the label-free images, and also the processing of labeled images into cell feature maps 2122. The alignment and registration may be done using fiducial markings on the consumable that are imaged/located by both the main imager 2104 and the secondary imager 2116. For this purpose, the secondary imager 2116 may also employ a transmission (label-free) imaging channel. In some cases, label-free images from the main imager 2104 are provided to the second computing subsystem 2120, and image registration is performed based on cell culture features, rather than on a co-aligned global X, Y coordinate system. Cell- or cell cluster-level registration allows for more accurate registration in some cases, particularly when there have been some shifts in the biology between the two imaging modalities, or where no fiducial marks are present on consumables.

In some implementations, such feature-based registration may be done with established methods for extracting key points (potentially at multiple scales) and transforming geometries between images, to map the labeled data onto the coordinates of the label-free data. This process may be performed at the image level, or at the output map level, for example re-mapping cell phenotype based on fluorescent protein expression based on a geometric mapping between the transmission image co-captured with the fluorescent imaging, and the transmission image captured by the high-throughput imager.

In other implementations, such feature-based registration may be done through a deep learning model which outputs the parameters of a transformation matrix that aligns given sub-regions (patches) of the label image to corresponding sub-regions of its label-free transmission light version captured by the second imager 2116 (along with all the other associated fluorescent artifacts). Such an alignment deep learning model may be trained using a specially acquired and prepared training dataset. Specifically, two label-free images can be acquired by the same imager with a time-resolution that roughly corresponds to the time-resolution of images acquired by the two imaging systems (e.g., to match the temporal difference between label-free images from imager 604 and label-free images from imager 2116). These two acquired images would have perfect pixel-level alignment as they would be acquired by the same imager without moving the container in between acquisitions. However, the biology may shift and change as would be expected due to cell growth, death, and divisions, hence the image features wouldn't be in perfect alignment.

One of these two images would then be further subjected to a global affine transformation to simulate a warping due to a slight 3D shift and rotation of the cell culture container when it is delivered to a nest of an imager. A deep learning model would then be trained to regress this global affine transformation's parameters given two patches from the input images. Note that the patches need to be sampled from feature-rich areas of the two images. In this way, the noise introduced by changing biology in between image acquisitions would be captured in this training set and the model would learn to regress an accurate affine model despite such changing biology. Furthermore, during inference, multiple patches from the two images can be used to estimate affine matrices and a random sample consensus (RANSAC) procedure may be used to finalize a global affine matrix for the given pair of images. This global matrix may be used to initialize a refinement procedure such as a Levenberg-Marquardt optimization algorithm which uses the full images and a dense matching metric such as a correlation score to further adjust the alignment parameters. Note that the above alignment techniques may be applied to sub-images of a container image independently and in a way that optimizes the alignment of the respective sub-image features only. Such partitioning of the full cell culture container image into sub-images may be particularly relevant when a global linear alignment of the whole container image is not possible due to non-linear warping effects in between imaging sessions, and works well in the case when locally linear alignments may be computed efficiently via the disclosed methods. In such cases, care should be taken to create sub-images with some overlap margins and blend the margins as corrected sub-images are stitched back together to form a container-level aligned image.

Once there are aligned images from imagers 2104, 2116, resulting cell culture maps 2122 may then be used to train the deep learning model(s) by the first computing subsystem 2110, via training or retraining system 2124. A selection system 2126 may be used to selectively pick patches for training within this aligned ground truth image collection, based on their features and/or the embeddings of corresponding label-free images. The training system ultimately provides or updates model weights 2112 for the deep learning model(s), which generates cell feature maps from label-free images alone. The model(s) may take single time point label-free images, or may take a time series of label-free images to generate these maps. The use of X, Y, Z-aligned time series, for example, may provide not only morphological information, but also dynamic information with regards to cell proliferation, death, migration, and/or morphology changes that may be key to mapping phenotype and other characteristics.

### High Throughput High Resolution Bio-Driven Spatial Phenomics

Patient-specific cell manufacturing workflows are followed by expensive release assays which also require expansion of cells beyond the therapy doses and delay the delivery of the product. It would be beneficial to replace as many of the end-process assays with in-process assays where the manufacturing platform's imagers can be used as the main sensing modality. Normal imaging resolution of the manufacturing workflows may not be high enough to capture various cell phenotypes such as cell shapes and their organelles' spatial and temporal feature distributions. A high information content only provided by higher magnifications than the normal manufacturing magnification may be required for an AI system to reliably learn the correlations of cell phenotypes to various conditions that are screened through traditional assays such as abnormal karyotype or expression of certain diseased genomic profiles. Thus, there is a need in the art for improved methods of collecting image-based QC assays mid-run of a cell culture process.

Systems and methods disclosed herein include an imaging subsystem with multiple magnification options and the ability to adjust magnification level dynamically for a given acquisition, along with an AI-driven control system. The control system is configured to create a stack of varying resolution images to correspond to the regions of a cell culture container where cells are growing. Specifically, multiple passes of acquisitions at an increasing level of magnification would be coordinated by an AI-driven controller where each layer of imaging is processed by the AI engine to create a target subset of tiles to be acquired at the next magnification level. A multi-resolution image stack may be generated through an AI controller analyzing the biological content of a cell culture container from the current imaging layer and deciding on a new target set of tiles to be captured in the next magnification level. In an example, a base 2× image is captured to initiate the process covering the full cell culture container. Then a series of 4×, 10×, and 40× acquisitions are run at target tiles determined by the AI controller.

Such a system would be capable of creating a stack of images with, for example, a base resolution of 2× magnification and a varying stack of 4× to 40× resolution tiles that cover the selected regions of the cell culture container where biologically interesting features are observed and a finer inspection is needed. The cell culture system's UI may be configured to dynamically fetch the higher resolution images as the user zooms into a given location within the cell culture container. In this way, the user would start by observing a cell colony at 2X, and zoom all the way down to an individual cell and its organelles at the highest magnification, e.g., 40X. The UI may be configured to provide indicator boxes overlaid on the base image showing the availability of high resolution data. For a smooth zooming experience, the system may be configured to interpolate resolution levels between magnification level changes.

Given such a stack of images, the AI engine may be configured to predict the existence of various conditions such as abnormal karyotype at the required resolution of that condition. A plurality of deep learning models may be trained from a large collection of such dynamically collected multi-resolution image stacks. In such a training dataset, the biological samples may be carefully curated from samples with known conditions or with normal passing release assays. The optimal resolution for prediction of a particular condition may be determined through comparative model performance analysis. The best models at the corresponding resolution may be deployed to the AI engine of the cell culture system.

During manufacturing, the cell culture system may include one or more of such imagers capable of bio-driven magnification adjustments and the cell culture containers may be imaged at selected time points by such imagers for in-process quality control of the samples. Such bio-driven imaging would take less time than acquiring images of the full cell culture container at each magnification level and enable high resolution inspection of cell phenomics by the AI engine.

### Image-Based Anomaly Event Detection During Adherent Cell Manufacturing

The culturing of iPSCs may be compromised by various anomaly events. Early identification of these anomalies is crucial to minimize investment in failing cell culture processes, improve laboratory processes, and optimize material selection. An example of an anomaly that should be detected includes early detection of fungal and bacterial contamination, in which it is important to implement techniques to identify contamination as early as possible to prevent its spread and ensure quality control. Another example of an anomaly that should be detected is identification of chamber defects, such as detecting defects in cell culture chambers caused by hardware issues. Another example of an anomaly that should be detected is detection of anomalous cell growth, in which identifying cell growth patterns that deviate from normal cells and clusters would enable early intervention and corrective actions. Thus, there is a need in the art for dynamic, in-process anomaly detection during a cell culture process.

Systems and methods disclosed herein include an image-based anomaly monitoring system in which cell culture containers are imaged by the cell culture system's imaging subsystem periodically as part of various bioprocesses and an anomaly detection inference system evaluates each new image. The system builds various indices from a collection of training image data during a training phase for fast inference during the monitoring phase.

During the training phase, a training pipeline may be constructed to extract the image embedding vectors from a large collection of normal images, as well as from smaller collections of known categories of abnormal images such as fungus images, bacteria images, anomalous cell growth images, chamber defect images, etc. The image embedding vectors may be extracted using a backbone deep learning model which is trained as a feature extractor in a self-supervised way on a large collection of label-free images captured by the system's imaging subsystem. Once embedding vectors are available, the next stage is coreset building. A coreset is a subset of embedding vectors that is representative of the distribution of vectors in the latent embedding space. In this stage, a coreset selection step is applied to extract the coreset from the normal embeddings. For example, the coreset may be extracted from the fungus embeddings, bacteria embeddings, anomalous cell growth embeddings, chamber defect embeddings, and so on for any other known anomaly category. For each of the coresets, the system then runs a search index builder, resulting in a normal search index, fungus search index, bacteria search index, anomalous cell growth search index, and chamber defect search index. The same workflow may be used to generate search indices for any other anomaly category.

During the monitoring phase, for a given new image, the monitoring subsystem determines if there are preceding images from the same cell culture chamber which are not already tagged with any abnormalities. If so, the monitoring subsystem extracts embeddings from the preceding images using a backbone feature extractor, generates a preceding coreset using the coreset selection method, updates the normal coreset by adding the preceding coreset, and updates the normal search index which is now customized to the new image. Then the monitoring subsystem extracts the embeddings of the new image using the same backbone feature extractor and calculates the distance from the new image embeddings to the normal coreset. If the largest distance is no larger than the anomaly threshold, the system tags the new image as normal. Otherwise, the system calculates the distance from the new image embeddings to each anomaly coreset by the corresponding search index, and tags the image as anomalous with the closest anomaly coreset name.

### Systems for Adaptive Laser Processing of Cells

Laser-initiated microbubble cell processing, accomplished using a laser and an absorbing target or targets in the vicinity of cells, may include: temporary poration of cell membranes to allow intracellular delivery or extraction of material from cells; detachment of cells from a surface; and/or terminating cells for the purpose of controlling a cell culture (e.g., managing cell density, enriching a cell population, selecting a clonal population, etc.). In the case of cell termination, a range of mechanisms may be employed including but not limited to cell lysis, or damage to cell membrane and/or internal components to the point at which cell death occurs by apoptosis or necrosis. Laser pulse absorption may be accomplished by plasmonic nanoparticles in the cell culture/media, or a laser-absorbing film on the surface of the cell culture container.

The amount of mechanical energy required to achieve the desired effect on cells may vary greatly depending on a number of factors including cells (e.g., cell density, cell size, cell phenotypes), media, extracellular matrix (ECM) state, and laser absorber (e.g., laser-absorbing particle concentration and/or composition, or laser film absorption and thermal properties). Simultaneously, it may not be a viable strategy to simply address these variations by applying excess laser energy to the system. Excess energy may kill cells that were not meant to be destroyed (e.g., kill cells when attempting to perform intracellular delivery), cause collateral damage around targeted cells, increase local or global media/substrate/cell temperatures, and/or damage laser absorbers such as nanoparticles or laser absorbing film. Balancing these two factors may lead to incomplete cell processing as intended, and as a result, deviations from the planned bioprocess. Thus there is a need in the art for precise and adaptive control of laser energy in an optical-based bioprocess.

Systems and methods disclosed herein include those for adaptively tailoring laser pulse energies, pulse rates, spacing, etc. to achieve desired effects on cells while minimizing the off-target effects on cell cultures or bioprocesses. Systems and methods for immediate measurement of laser processing effects on cells using imaging prior to and after such laser processing are described herein. This method may also result in maps of successfully treated versus unsuccessfully treated cells. Alternatively, after additional operations such as washing, there may be mapping of successfully versus unsuccessfully treated regions.

Laser scanning parameters that may be varied in the present implementations include, but are not limited to, pulse energy (for example, modulated by an acousto-optic modulator to a specific pulse energy), pulse rate, pulse spacing (e.g., microns between pulse hit locations), pulse spot size (the diameter of the spot illuminated by the laser pulse on the absorber, which may be controlled by focus, or by an additional optical element such as a fast-focusing lens), pulse repetitions on the same location, and pulse length (e.g., in the extreme case, nanosecond or sub-nanosecond pulses in some cases, to continuous-wave illumination in others). In some implementations, pulse spacing along different axes may be controlled independently, for example wide spacing along one axis and narrow spacing along another. Pulse spacing does not need to be at regular intervals along each axis, for example when allowing for clusters of laser hits that may better preserve ECM coating function while still killing and/or dislodging cells.

These laser parameters may be generated to match laser scan patterns in a raster map or vector form, and may be varied point-by-point (laser pulse to laser pulse), by region/segment, or on entire scans. The laser parameters may be driven dynamically by factors including but not limited to: cell density maps, for example local cell density within a cell colony or culture; cell phenotype prediction maps, for example generated from a deep learning model; stage of progression through a cell bioprocess; local or global state of ECM (for example, time elapsed since deposition of the ECM, time elapsed with cells growing on ECM, number of times ECM has been laser-scanned locally, or based on mapping of ECM coverage, either a one-time measurement for a container and protocol, or a measurement performed during every ECM coating); previous results of laser scanning a cell population, including but not limited to a particular patient source, clonal population, batch, colony, region within a container, or population within a container (such results may be computed from pre- and post-scan imaging, imaging after washing, and/or imaging after a specified time period); cell morphology maps; maps based on an embedding vector generated per local patch from cell culture images; maps of cells versus direction of liquid flow; for example position on leading or trailing edge of a cell colony with respect to liquid flow in a fluidic chamber; location within a cell culture container, for example radial location in a circular well, or lateral or longitudinal location in a fluidic chamber; and maps of cell layer thickness or 3D characteristics of cells in an adherent cell culture, for example a map that shows cell detachment from the substrate, or a map that shows regions where cells are growing on top of one another.

### Closed-Loop Cell Washing Using Imaging

Unit operations such as liquid washes, lasers, cell seeding, or other processes are applied to cells grown in a closed cell culture chamber. These unit operations result in changes in cells in the cell culture chamber, including adding or removing cells. The end result of these unit operations may be imaged by placing the chamber under a microscope after the process. Unexpected interactions or errors cannot be seen as they occur. One example is an attempt to remove cells from the cell culture chamber resulting in the collection of those cells in a clump elsewhere in the chamber. The dynamics of this issue resulting in the clump are not known. Thus there is a need in the art for tracking changes to a cell culture during washing and adjusting for those changes.

Systems and methods disclosed herein include a real-time system for imaging of unit operations inside a cell culture chamber. For example, during cell washing, fluid streams across the cell growth surface in the cell culture chamber. Real-time imaging captures changes at each time point, t, during the washing process. Tracking of cells as they are washed away can indicate how uniformly cells are removed at each time point. Imaging may also reveal the formation of unexpected structures, such as clumps of cells forming in an unexpected location.

The operations supported herein include, but are not limited to, imaging of unit operations while they are underway, triggering of image capture based on input/output signals or other hardware triggers, synchronized capture of image frames with sensor readings to create a single timeline, selection of region of interest (ROI) to image, specifying a list of actions (e.g., image ROI, triggers, sensor synchronization, duration), tracking changes in pixel density in the ROI, tracking moving objects in the ROI, and streaming images to storage for later analysis. An imaging run during a unit operation results in a timeline of images along with synchronized sensor and trigger data for each time point (e.g., image, sensor_0, sensor_1, . . . sensor_n). In this way, imaging may be used to correlate changes in cells being imaged with sensor readings at each time point.

### Active Cell Expansion Management

The present implementations cover methods for pre-patterning cells prior to passaging (i.e., transferring from one cell growth container to another), including through the use of cell removal tools (CRTs) as described herein, including but not limited to laser-based tools. The present implementations additionally cover methods for managing colonies within cell culture containers, including but not limited to fluidic growth chambers and cassettes. These methods include methods for expanding selected colonies in an efficient manner that is compatible with closed fluidic cassettes, including methods for controlled mini-expansion of select colonies to ensure consistent and healthy cell densities while growing the total number of cells. Additionally, methods are disclosed for optimally efficient cell expansion (e.g., maximizing expansion rate per passage step) and optimal use of growth surface, and methods for spatial patterning prior to differentiation steps.

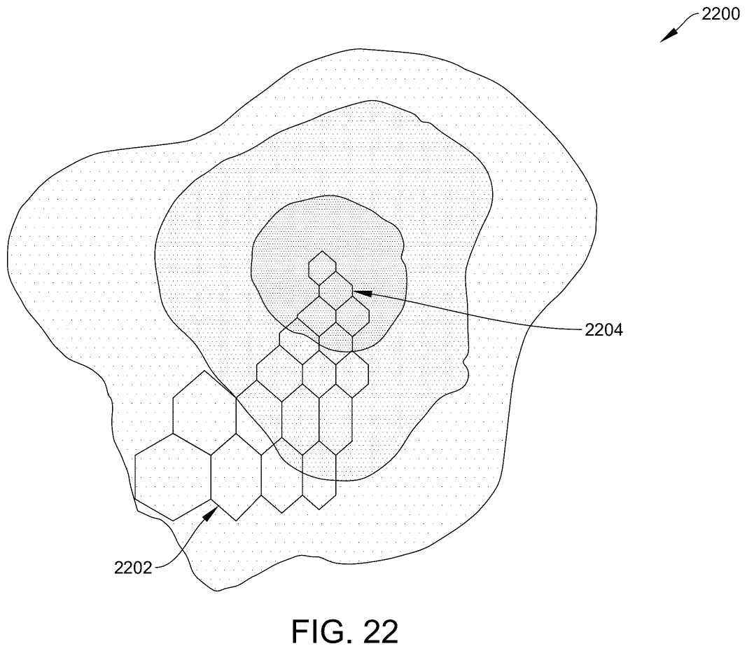

FIG. 22 is a diagram illustrating a method of pre-patterning cell colonies in accordance with various implementations. The pre-patterning may be performed by cutting all or a portion of a cell colony 2200 into patches, each containing a roughly equal number of cells. The cell colony 2200 may contain regions of varying cell density. The goal of the pre-patterning method is to provide cell clusters of roughly equal cell count for downstream processes, such as seeding onto a new surface or culturing in suspension. The cell colony 2200 may be cut, by a CRT in a grid pattern, into larger islands 2202 in low-density regions and smaller islands 2204 in high-density regions, resulting in clusters of roughly consistent cell count. A variety of patterns may be used to provide a combination of cell health (e.g., leaving cells with the appropriate number of neighbors, such as more circular or hexagonal patches if a large number of neighbors is desired), minimizing “kerf loss” (i.e., the width of material removed by the CRT) (for example, kerfs of ≤100 microns, or ≤75 microns, or ≤50 microns or even ≤25 microns), and maximizing the ease of harvesting the resulting islands using chemical and/or mechanical means.

In some implementations, the CRT cut lines may advantageously leverage mechanical forces of cell-cell interactions or cell growth patterns. For example, if cells are elongated and directional, islands that are longer in the longer axis of the cells may be cut (analogous to cutting along the grain of a material). The CRT used to pattern the cell colony may include, but not be limited to, a laser system that uses a pulsed laser to interact with a semi-transparent film on which the cell growth occurs. In some implementations, a wait time is imposed between when the cell sheet is patterned and when the cell clusters are harvested, to allow cells to become more dense within the patterned islands. Such densification, which may improve intact removal of the islands, may be promoted by CRT removal of the extracellular matrix (ECM) in the cut lines. In some implementations, the pattern may be applied multiple times to kill/remove cells and/or ECM while keeping the individual cut actions as stress-free as possible for the surrounding remaining cells.

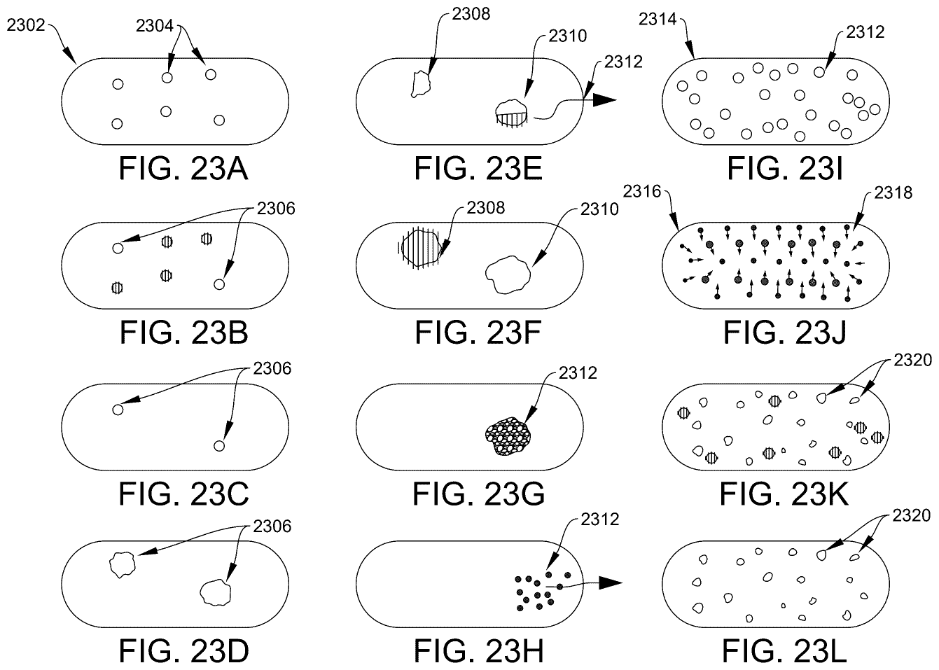

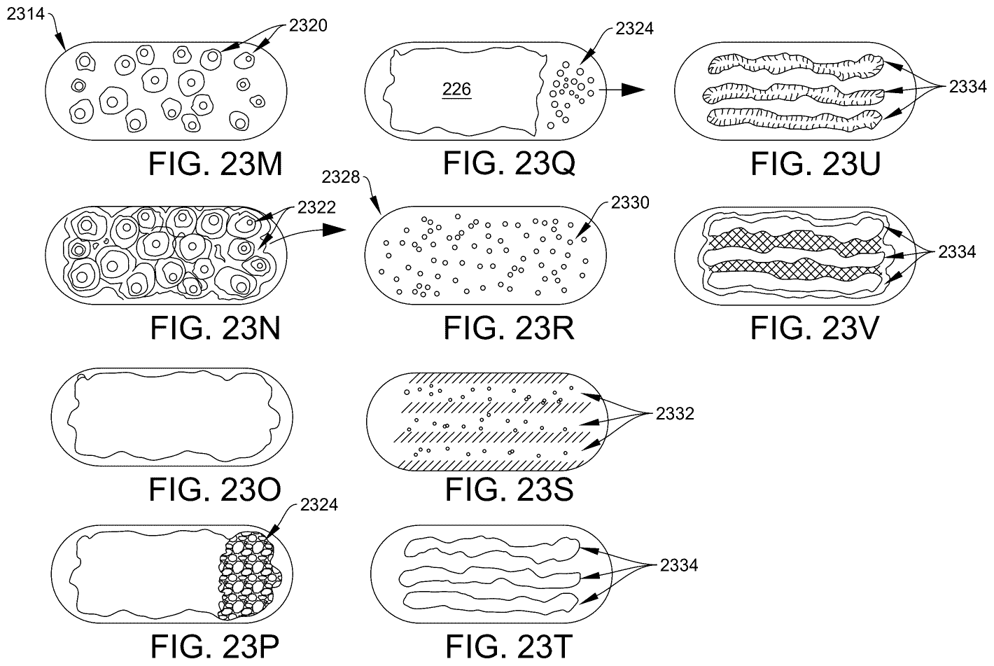

FIGS. 23A-V are diagrams illustrating an example series of cell culture process operations in accordance with various implementations. FIG. 23A depicts a cell culture container 2302, which may be a fluidic chamber (e.g., a fluidic closed cassette), containing a plurality of cell colonies 2304 growing on a cell culture surface of the container 2302. The cell colonies 2304 may be, for example, clonal induced pluripotent stem cell (iPSC) colonies that have been managed on the cell culture surface for an extended period after reprogramming and/or gene editing to stabilize the cells and clear any reprogramming vector. This maintenance may include feature tracking that is used to predict clonal quality and rank the clones, as described herein. In the case of reprogramming, these colonies may have grown directly out of the reprogrammed material, either in the same cell culture container or previously passaged from another “early emergence” cell culture container. In some implementations, multiple separated cell colonies 2304 may be density-managed by CRT at healthy local cell densities, for example at colony densities of ≥1/cm², ≥2/cm², or ≥5/cm².

FIG. 23B depicts removal by a CRT of most of the cell colonies shown in FIG. 23A, except for the top two clonal cell colonies 2306. The cell colonies 2304 may be ranked based on predictions of colony quality, either by a machine learning model or by human inspection, or a combination of automated and manual evaluation. In the example shown in FIG. 23B, only two cell colonies 2306 are retained, but in general a certain percentage of colonies may be retained after CRT removal. For example, less than 50% of cell colonies may be retained, or less than 25%, or less than 10%. FIG. 23C shows the retained cell colonies 2306 after cell debris (i.e., the other cell colonies removed from the cell culture surface) are washed away. FIG. 23D shows the retained cell colonies 2306 after a period of time when they are allowed to expand in area and cell count. The growth characteristics of the clonal colonies 2306 may be tracked for later use, for example using an imaging subsystem and a computing subsystem of a cell culture system. Methods of tracking cell growth and dynamics such as those described herein may be used to track behavior of each colony.

FIG. 23E depicts the cell culture container 2302 after a portion of a second-ranked cell colony 2308 has been removed to maintain healthy cell density (and debris has been cleared from the cell culture container) and a portion of a top-ranked colony 2310 is removed by the CRT and the resulting cell debris 2312 is retrieved from the cell culture container 2302 for analysis. The analysis may include but is not limited to genomic integrity testing, DNA sequencing, RNA sequencing, proteomics, etc. The purpose of the analysis may be to confirm the quality of the top-ranked colony 2310, or to indicate an issue with it and proceed with the second-ranked colony 2308. FIG. 23F depicts removal of the second-ranked cell colony 2308 using a CRT, leaving the top-ranked cell colony 2310 (the quality of which may be confirmed by the harvested debris assay). The debris from the second-ranked cell colony 2308 may be washed away from the cell culture container 2302.

FIG. 23G depicts the selected top-ranked cell colony cut into clusters 2312. The clusters 2312 may be cut to each contain a target number of live cells, as described with reference to FIG. 22. The target cell count per cluster may be previously determined by experiments harvesting these clusters from the cell culture container 2302 and seeding the clusters onto a new cell culture surface, and analyzing the clusters in terms of survival rate and/or target cluster size in the downstream cell culture container. FIG. 23H depicts harvesting of the clusters 2312 out of the cell culture container 2302. The harvest may be performed using chemical and/or mechanical means. In some implementations, the pre-cutting of the clusters 2312 using the CRT may facilitate the harvest, or improve the health of the harvested clusters.

FIG. 23I depicts a new cell culture container 2314 into which the clusters 2312 have been seeded. The clusters 2312 attach to the cell culture surface of the cell culture container 2314 and proliferate. Through imaging and computer vision models (including but not limited to deep learning models), the position, area, and area growth rate and directionality (derived from time-series images), if any, may be determined for each cluster. FIG. 23J depicts a map 2316 of the cell culture container 2314 based on previous cell expansion observations. The map 2316 shows the average cell expansion rate as well as directionality, as represented by the point sizes and arrows 2318, respectively. Spatial variations in these characteristics may result from a range of effects, including non-uniformities in the ECM, non-uniform nutrient distribution or waste removal in cell media, non-uniform dissolved gas concentrations, non-uniform effects of fluid flow profiles, spatially varying mechanical or thermal variations, etc. Local cell/colony behaviors may have been tracked via methods described herein for back-tracing and characterizing cell dynamics.

FIG. 23K depicts selective removal of a subset of clusters based on a map computed to achieve the highest uniformity of cell confluence at the end of cell culture expansion, leaving retained clusters 2320. The map may be computed from information on the overall clonal behavior observed in prior steps of the process (e.g., area proliferation rate, cell proliferation rate, cell division rate as a function of cell density, etc.), the cluster-specific features observed after seeding and initial growth, and/or the spatial map 2318 of past cell expansion characteristics. The map may be generated from a heuristic model, from a series of simulations, or from a reinforcement learning model that has been trained on prior runs and/or simulated runs. In some implementations, clusters may be partially removed to reduce cell count, and therefore tune when a cluster is expected to achieve a target size. In other implementations, the ECM may be selectively removed from regions around a cluster to direct/shape the growth of the cluster. The objective of all the actions performed by the CRT is to set up the earliest stage of expansion to achieve the most uniform possible growth over a large part of the cell culture container 2314.

FIG. 23L depicts the cell culture after the selective cluster removal shown with reference to FIG. 23K, leaving the retained clusters 2320 to proliferate. The retained clusters 2320 may have been modified by the CRT and/or the surrounding regions may be modified by the CRT, for example, having ECM partially or completely removed. FIG. 23M depicts the cell culture after proliferation, showing enlarged retained clusters 2320. FIG. 23N depicts the cell culture as it approaches maximum confluence. As the culture approaches confluence, some regions 2322 (often corresponding to the original cluster centers) may become over-dense. In such a case, the CRT may be used to remove the over-dense regions 2322 so that these regions do not spontaneously detach or differentiate. An arrow indicates that during such a de-densifying procedure, the resulting detached cells or cell debris may be collected for external analysis, as described herein. Additionally, the CRT may be used to remove regions showing abnormal or undesirable characteristics or dynamics, whether identified by cell or colony morphology, or cell/colony dynamics as tracked by tools described herein or similar.

FIG. 23O depicts the cell culture after further expansion which, due to cell culture management using the CRT from the start of the expansion, may achieve a high confluence without the formation of over-dense regions. Cells may be maintained at healthy densities, for example ≤5000 cells/mm², ≤5000 cells/mm², or ≤4000 cells/mm² locally for pluripotent stem cells, and with high overall confluence over the overall growth area, for example ≥70% confluence, ≥80% confluence, ≥90% confluence or ≥95% confluence. This ensures maximum area efficiency while maintaining uniform, healthy cell environs.

FIG. 23P depicts the expanded cell culture after it has been cut into islands/clusters 2324 with a CRT. FIG. 23P shows a scheme in which the cell culture sheet is scored in a manner that minimizes the stress and inconsistency in harvesting according to local features. The local features may include, for example, cell density, how long cells have been resident on the ECM at a particular location, and/or proximity to the edge of a contiguous cell sheet. All of these factors may impact the level of attachment to the cell culture container surface, and the requirements for harvesting them successfully. In this manner, the conditions for optimal harvesting are made more uniform, and less stressful (e.g., lower concentrations and/or shorter durations in dissociation agents, less mechanical action or fluidic shear stress required, etc.). In addition, the harvesting pattern shown in FIG. 23P is a partial harvest of the cell culture, defined at least in part by the CRT pre-cutting of the cell sheet (in some implementations, it may also involve spatially-varying application of reagents and/or fluidic/fluidic-air shear stresses). This partial harvest may be performed, for example, to achieve a specific concentration of cells/cell clumps per volume of liquid. This may be advantageous for harvesting in fluidic components (tubing, pipettes, valves, etc.) without clogging. This may also be advantageous for the purpose of achieving a target cell concentration for downstream processes, such as re-seeding.

FIG. 23Q depicts the now detached cell clusters 2324 being harvested out of the cell culture container 2314, while the remainder of the cell culture 2326 remains adhered in the cell culture container 2314. FIG. 23R depicts a new cell culture container 2328 into which the harvested cell clusters 2324 from cell culture container 2314 are seeded. The seeded cells 2330 may be seeded as single cells, for example, in preparation for a differentiation protocol to follow. FIG. 23S depicts a patterning applied via CRT to the cell culture container 2328 to, for example, prepare for a high-yield differentiation protocol resulting from better spatial control or to control the cell proliferation rate that is optimal for differentiation. Cells are allowed to remain in several linear regions 2332, while the cells that seeded in the areas between these are removed by the CRT. In addition, the non-cell areas may be fully or partially treated with the CRT to remove ECM. The patterning of the cells and ECM may be driven by previously observed growth characteristics of the source cells that have been seeded into the cell culture container 2328, on a time series observation of the initial growth of these cells, on known growth patterns in the cell culture container 2328, and/or on differentiation characteristics predicted (or previously measured) for the particular cell line, clone or patient.

FIG. 23T depicts the seeded cells after expansion into cell regions 2334, arranged in a manner to support good source cell health (for example, a sufficiently wide band of pluripotent cells to support good pluripotency), but also as to be optimally set up for differentiation, which in the example shown in FIG. 23T occurs in a spatially-dependent manner. The cell culture may be monitored via imaging by an imaging subsystem of a cell culture system. Based on maps of the cells computed from these images, and potentially previous cell behavior and/or assay data, the differentiation process may be initiated, for example, with the change of the cell media and its constituents. FIG. 23U depicts the cell regions 2334 undergoing initial differentiation around the edges of the patterned regions. FIG. 23V depicts the next stage of differentiation, in which the cell regions 2334 between the initially formed islands have differentiated into the target cell type. Thus, FIGS. 23A-V depict an example of utilizing CRT patterning, including patterning determined using a predictive model, to enhance the efficiency of cell differentiation processes.

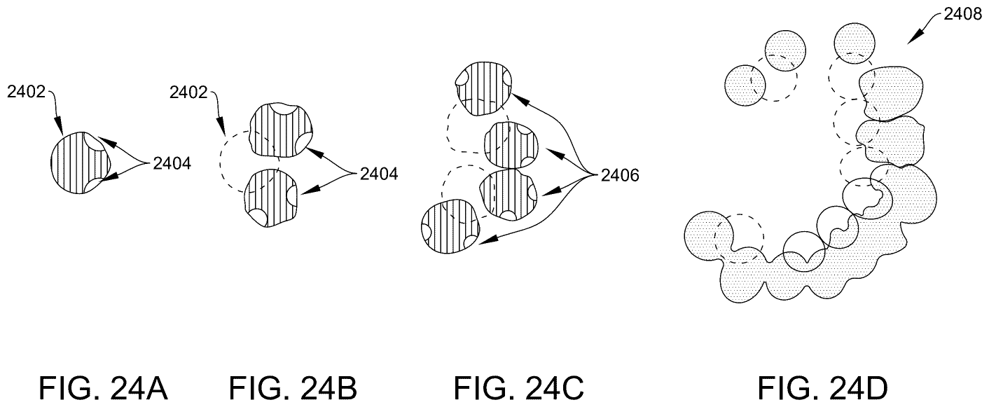

FIGS. 24A-D are diagrams illustrating an example method of an in-place expansion sequence for a cell colony in accordance with various implementations. An initial cell colony 2402 growing on a cell culture surface is reduced into two sub-colonies 2404 using a CRT. This allows a healthy low cell density to be maintained, and if ECM is destroyed/removed by the CRT, cells are translated towards regions with fresh ECM. FIG. 24B depicts proliferated colonies 2406 that grew out of the sub-colonies 2404, as well as an outline of the initial cell colony 2402. The proliferated colonies 2406 may be allowed to proliferate until the total area and number of cells have approximately doubled from the initial colony 2402. Additional CRT operations may be applied to each colony 2406 similar to the CRT operation performed in FIG. 24A, resulting in two smaller sub-colonies for each colony 2406 (or four sub-colonies in total).

FIG. 24C depicts another iteration of proliferation and CRT removal of the four sub-colonies generated in FIG. 24B. This yields four colonies 2406 that are each approximately the size of the initial colony 2402, each with healthy cell densities. CRT operations may be applied again to the colonies 2406, leaving two sub-colonies per colony. FIG. 24D depicts the result of another iteration of proliferation, in which the eight sub-colonies from FIG. 24C have formed a ring structure 2408. Thus, by repeated iterations of expansion and CRT removal, the initial colony 2402 has been proliferated significantly while (a) maintaining healthy cell density throughout; (b) not involving cell passaging or other disruptive steps that force re-plating of cells; and (c) moving the cells in a manner that allows one-time use of ECM surface for cell growth (i.e., the sub-colonies are translated relative to their parent colonies and proliferate into new areas of the cell culture surface where CRT operations have not damaged the ECM).

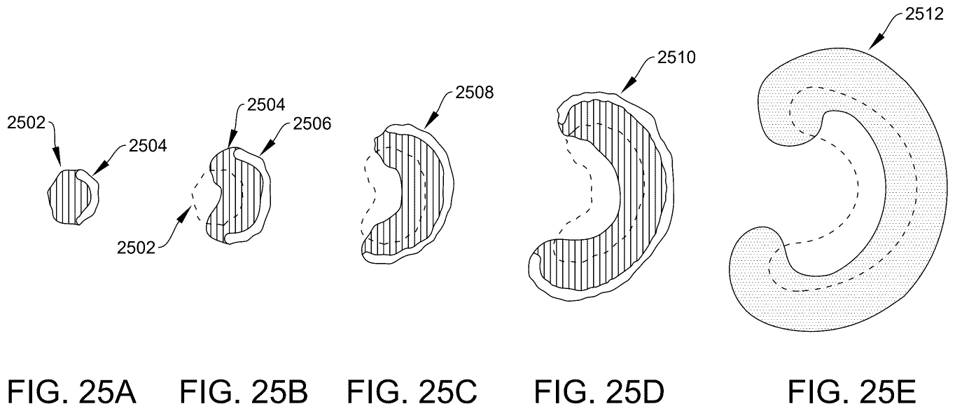

FIGS. 25A-E are diagrams illustrating another example method of an in-place expansion sequence for a cell colony in accordance with various implementations. FIG. 25A depicts an initial cell colony 2502 growing on a cell culture surface that is reduced in size by a CRT operation, leaving a crescent-shaped sub-colony 2504. The sub-colony 2504 may be crescent-shaped to encourage outward growth from the cell removal region, particularly if the ECM is removed by the CRT during cell removal. FIG. 25B depicts outlines of the initial colony 2502, as well as the sub-colony 2504 after expansion. Another CRT operation may be applied to the expanded sub-colony 2504, leaving another crescent-shaped sub-colony 2506 that is larger than sub-colony 2504. FIGS. 25C-D show additional iterations of proliferation and CRT removal operations, resulting in increasingly larger sub-colonies 2508 and 2510, respectively.

FIG. 25E depicts a final iteration of proliferation and CRT removal that results in a crescent-shaped colony 2512. Thus, the initial colony 2502 has been proliferated significantly while (a) maintaining healthy cell density throughout; (b) not involving cell passaging or other disruptive steps that force re-plating of cells; and (c) moving the cells in a manner that allows one-time use of ECM surface for cell growth (i.e., the sub-colonies are translated relative to their parent colonies and proliferate into new areas of the cell culture surface where CRT operations have not damaged the ECM).

While FIGS. 24A-D and 25A-E show example CRT removal patterns and retained sub-colony shapes (e.g., circular or crescent-shaped), in general any sub-colony shape and removal patterns are contemplated herein. For example, in some implementations the ECM may not be damaged by CRT operations or the ECM may be refreshed after CRT operations. This allows additional proliferation and removal iteration strategies. In one example, the CRT operation may remove the dense center of a circular cell colony, leaving a ring-shaped sub-colony that is allowed to proliferate both outwardly onto fresh ECM and inwardly towards the center onto the undamaged or refreshed ECM.

During in-place colony expansion, the tracking and dynamics measurement methods described herein may be used to collect features of the colony, for example cell motility, cell proliferation rate, density, etc. that may be used to characterize or rank colonies. In some implementations, this process may be combined with modulation of conditions to further elucidate colony state, phenotype, quality, viability, etc. Furthermore, the measured characteristics may be adjusted for location within the cell culture container as described herein.

### Other Considerations

While various implementations have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the implementations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings are used. Those skilled in the art will recognize, or be able to ascertain using no more than routine experimentation, many equivalents to the specific inventive implementations described herein. It is, therefore, to be understood that the foregoing implementations are presented by way of example only and that, within the scope of the appended claims and equivalents thereto, implementations may be practiced otherwise than as specifically described and claimed. In addition, any combination of two or more such features, systems, aspects, articles, materials, kits, and/or methods, if such features, systems, aspects, articles, materials, kits, and/or methods are not mutually inconsistent, is included within the inventive scope of the present disclosure. Particularly, any element of the disclosure and any aspect thereof may be combined, in any order and any combination, with any other element of the disclosure and any aspect thereof.

The above-described implementations can be implemented in any of numerous ways. For example, the implementations may be implemented using hardware, software, or a combination thereof. When implemented in software, the software code can be executed on any suitable processor or collection of processors, whether provided in a single computer or distributed among multiple computers.

As used in any implementation herein, a “circuit” or “circuitry” may include, for example, singly or in any combination, hardwired circuitry, programmable circuitry, state machine circuitry, and/or firmware that stores instructions executed by programmable circuitry. An “integrated circuit” may be a digital, analog, or mixed-signal semiconductor device and/or microelectronic device, such as, for example, but not limited to, a semiconductor integrated circuit chip.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone, or any other suitable portable or fixed electronic device. Also, a computer may have one or more input and output devices. These devices can be used, among other things, to present a user interface. Such computers may be interconnected by one or more networks in any suitable form, including a local area network or a wide area network, such as an enterprise network, an intelligent network (IN), or the Internet. Such networks may be based on any suitable technology and may operate according to any suitable protocol and may include wireless networks, wired networks, or fiber optic networks.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

Implementations of the methods described herein may be implemented using a processor and/or other programmable device. To that end, the methods described herein may be implemented on a tangible, non-transitory computer-readable medium having instructions stored thereon that, when executed by one or more processors, perform the methods. The computer-readable medium may include any type of tangible medium, for example, any type of disk including floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, magnetic or optical cards, or any type of media suitable for storing electronic instructions.

The terms “program” or “software” are used herein in a generic sense to refer to any type of computer code or set of computer-executable instructions that can be employed to program a computer or other processor to implement various aspects of implementations as discussed above. Additionally, it should be appreciated that according to one aspect, one or more computer programs that, when executed, perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various implementations. Also, data structures may be stored in computer-readable media in any suitable form.

Also, various inventive concepts may be embodied as one or more methods, of which an example has been provided. The acts performed as part of the method may be ordered in any suitable way. Accordingly, implementations may be constructed in which acts are performed in an order different than illustrated, which may include performing some acts simultaneously, even though shown as sequential acts in illustrative implementations.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. As used herein in the specification and in the claims, “or” should be understood to have the same meaning as “and/or” as defined above.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The term “coupled” as used herein refers to any connection, coupling, link or the like by which signals carried by one system element are imparted to the “coupled” element. Such “coupled” devices, or signals and devices, are not necessarily directly connected to one another and may be separated by intermediate components or devices that may manipulate or modify such signals. Likewise, the terms “connected” or “coupled” as used herein in regard to mechanical or physical connections or couplings is a relative term and does not require a direct physical connection.

Unless otherwise stated, use of the word “substantially” may be construed to include a precise relationship, condition, arrangement, orientation, and/or other characteristic, and deviations thereof as understood by one of ordinary skill in the art, to the extent that such deviations do not materially affect the disclosed methods and systems.

It will be appreciated by those skilled in the art that any block diagrams herein represent conceptual views of illustrative circuitry embodying the principles of the disclosure. Similarly, it will be appreciated that any flow charts, flow diagrams, state transition diagrams, pseudocode, and the like represent various processes which may be substantially represented in computer readable medium and so executed by a computer or processor, whether or not such computer or processor is explicitly shown. Software modules, or simply modules which are implied to be software, may be represented herein as any combination of flowchart elements or other elements indicating performance of process steps and/or textual description. Such modules may be executed by hardware that is expressly or implicitly shown.

We claim:

1. A cell manufacturing platform, comprising:
a cell culture cassette configured to support a cell culture;
an optical engine configured to:
capture images of the cell culture; and
optically remove cells from the cell culture, wherein
the optical engine is configured to move relative to the cell culture cassette to capture the images and to remove cells without movement of the cell culture cassette.

2. The platform of claim 1, wherein the optical engine comprises:
a first linear stage configured to move along a plane of the cell culture cassette; and
at least one light source mounted on the first linear stage.

3. The platform of claim 2, wherein the first linear stage is further configured to move perpendicular to the plane of the cell culture cassette.

4. The platform of claim 1, wherein the optical engine comprises:
a first linear stage configured to move in a first linear direction;
a second linear stage mounted on the first linear stage, the second linear stage configured to move in a second linear direction perpendicular to the first linear direction; and
at least one light source mounted on the second linear stage.

5. The platform of claim 4, wherein the optical engine is further configured to move perpendicular to a plane of the cell culture cassette.

6. The platform of claim 4, wherein the optical engine further comprises one or more actuators configured to independently move the first linear stage and the second linear stage, thereby moving the at least one light source relative to the cell culture cassette.

7. The platform of claim 1, wherein the cell culture cassette comprises a semi-transparent surface configured to support the cell culture.

8. The platform of claim 7, wherein the semi-transparent surface comprises an optical film configured for imaging and cell removal by the optical engine.

9. The platform of claim 1, wherein:
the optical engine further comprises one or more sensors configured to measure optical power of the optical engine, the platform further comprising:
a platform manager configured to adjust the optical power of the optical engine based on the measured optical power, thereby adjusting a precision of cell removal operations.

10. The platform of claim 1, wherein the cell culture cassette is located on a first side of a transparent box and the optical engine is located on a second side of the transparent box.

11. The platform of claim 10, wherein the platform is configured to adjust a laser energy based on a sterility requirement associated with the first side or the second side of the transparent box.

12. The platform of claim 1, further comprising a robotic system configured to move the cell culture cassette to and from the optical engine.

13. The platform of claim 12, further comprising a plurality of additional cell culture cassettes, wherein the robotic system is further configured to move each of the plurality of additional cell culture cassettes to the optical engine.

14. The platform of claim 1, wherein the optical engine comprises a laser light source configured to emit laser light to remove cells from the cell culture.

15. The platform of claim 14, wherein the laser light source is configured to emit laser light at a wavelength between 700 nm and 1000 nm.

16. The platform of claim 1, wherein the optical engine further comprises an imaging system configured to capture brightfield and fluorescence images of the cell culture.

17. The platform of claim 1, further comprising a platform manager configured to:
receive image data of the cell culture from the optical engine;
analyze the image data to identify target cells for removal; and
control the optical engine to remove the identified target cells.

18. The platform of claim 17, wherein the platform manager is further configured to:
generate a cell removal pattern based on the analyzed image data; and
control the optical engine to remove cells according to the generated cell removal pattern.

19. The platform of claim 1, wherein the cell culture cassette comprises a plurality of cell culture chambers, each chamber configured to support a separate cell culture.

20. The platform of claim 1, further comprising an environmental control system configured to maintain temperature, humidity, and gas composition within the cell culture cassette.

21. A method of cell manufacturing, comprising:
providing a cell culture cassette;
adhering a cell culture within the cell culture cassette;
capturing images of the cell culture by an optical engine;
optically removing cells from the cell culture by the optical engine; and
moving the optical engine relative to the cell culture cassette to capture the images and to remove cells without movement of the cell culture cassette.
